(19) United States
(12) Patent Application Publication
(10) Pub. No.: US 2005/0129617 A1
(43) Pub. Date: Jun. 16, 2005

(54) Type1 diabetes diagnostics and therapeutics

(76) Inventors: Rusung Tan, Vancouver (CA); Bruce C. Verchere, Vancouver (CA); Jacqueline Trudeau, Vancouver (CA)

Correspondence Address: GRAYBEAL, JACKSON, HALEY LLP, 155 - 108TH AVENUE NE, SUITE 350, BELLEVUE, WA, 98004-5901, US

(21) Appl. No.: 10/481,696

(22) PCT Filed: Jun. 25, 2002
(86) PCT No.: PCT/CA02/00975

Related U.S. Application Data
(60) Provisional application No. 60/299,754, filed on Jun. 22, 2001.

(51) Int. Cl. A61K 49/00; C12Q 1/00; A61K 38/10

(52) U.S. Cl. 424/9.1; 514/15; 435/4

(57) ABSTRACT

The invention provides compounds and methods useful for the diagnosis, prediction, therapy, or prophylaxis of type 1 diabetes. The compounds of the invention include peptides derived from IAPP (islet amyloid polypeptide) precursor peptides.

### FIELD OF THE INVENTION

The invention is in the field of autoimmune diseases. One aspect of the invention relates to peptide compounds that are relevant to diagnosis and therapy of type 1 diabetes.

### BACKGROUND OF THE INVENTION

Diabetes mellitus is a metabolic disorder characterised by insulin deficiency and consequent hyperglycaemia, which can result in blindness, cardiovascular disease, or kidney failure, and when acute, lead to diabetic coma or death.

Type 1 diabetes, previously sometimes known as juvenile diabetes or insulin-dependant diabetes mellitus (IDDM), is characterised by an autoimmune response in which specific T lymphocytes gradually destroy the insulin-producing beta cells of the pancreas. The initial phase of leukocyte infiltration into the beta cells is known as insulitis, and entails both inflammation and attack by cytotoxic antibodies. Insulitis is followed by the actual destruction of the beta cells. Overt clinical symptoms of diabetes are generally manifested only when over 90% of the beta cells are destroyed. The effects of loss of beta cells are dramatic; weakness, weight loss, vision problems, and excessive hunger and thirst are among the early symptoms of type 1 diabetes. Eventually, a type 1 diabetic patient is typically insulin-dependent for life. Even with regular insulin injections, type 1 diabetes can still reduce the life span of a patient by an average of twenty years, and severely impact the quality of life of both diabetic patients and their families.

It is generally accepted that the destruction of beta cells is largely mediated by cytotoxic T lymphocytes (CTL) that specifically recognise antigenic beta cell-derived peptides. CTLs (otherwise known as CD8+ T cells) are “killer” cells which play an important role in protecting mammals from viral infection. When CTLs recognise antigens through their specific T-cell receptor (TCR), they are activated to divide, differentiate, and kill infected cells. CTLs, through their TCR, recognise small peptides, in the context of major histocompatibility complex (MHC) class I molecules. MHC molecules (called human leukocyte antigens or HLA in humans) are extremely polymorphic, particularly in the peptide-binding groove. Different MHC molecules will therefore bind to different peptide sequences. The human MHC class I molecule, HLA-A*0201, is very common in humans, particularly Caucasians, who are the most susceptible population for developing type 1 diabetes.

It has been postulated that some autoimmune diseases, such as type 1 diabetes, occur when aberrant CTLs recognise proteins (auto-antigens) displayed by MHC class I molecules. In the case of type 1 diabetes, the aberrant CTLs are apparently activated to kill beta cells by recognizing particular peptide epitopes. In the non-obese diabetic (NOD) mouse, an animal model of type 1 diabetes, a number of peptide epitopes have been described. Peptides known as NRP and NRP-A7 are reportedly recognized by a T-lymphocyte population in the context of H-2Kd class I MHC molecules (Amrani et al., 2000). Similarly, a variety of insulin-derived peptides have been implicated as autoantigens in diabetes, (both MHC class I and class II) such as a peptide derived from the B chain of insulin (see Daniel and Wegmann, 1996; and Wong et al., 2001 and 2002). Recently, a peptide-based therapeutic called DiaPep277 derived from a heat-shock protein has been shown to be capable of immunomodulation in type 1 diabetes patients (Raz et al., 2001, Lancet 358: 1749-1753), and the use of other heat shock proteins as immune modulators in type 1 diabetes has been suggested (see U.S. Pat. No. 6,007,821 issued to Srivastava et al., 1999).

In keeping with the finding that immune system diseases may be caused by aberrant T cells, therapies have been implemented that seek to selectively eliminate or reduce the levels of a particular T cell population. For example, extracorporeal photochemotherapy (“photopheresis”) has been proposed for the treatment of cutaneous T cell lymphoma (Edelson, R., “Light-activated Drugs”, Scientific American 256(8): 68-75 (1988); Edelson, R., “Photopheresis: A Clinically Relevant Immunobiologic Response Modifier”, Annals of N.Y. Academy of Sciences 636:154-164 (1991). Such treatments may involve eliciting a specific response to the aberrant T cells that is mediated by T cell surface receptors. In effect, methods that allow the isolation of aberrant T cells may be used to prepare vaccines useful for vaccinating a patient against their own aberrant T cells (see for example U.S. Pat. Nos. 4,838,852 and 5,147,289, incorporated herein by reference). Photopheresis has accordingly been used for the treatment of several autoimmune disorders, including pemphigus vulgaris, systemic sclerosis and rheumatoid arthritis (Rook, A., 1991, “Photopheresis in the Treatment of Autoimmune Disease: Experience with Pemphigus Vulgaris and Systemic Sclerosis”, Annals of N.Y. Academy of Science 636:209-216; Malawista, S., et al., 1991, “Photopheresis for Rheumatoid Arthritis”, Annals of N.Y. Academy of Science 636:217-226). Similarly, it has been reported that animals may be vaccinated against autoimmune diabetes with a T-cell epitope of the human 65 kDa heat shock protein (Elias et al., 1991, P.N.A.S. USA v.88: 3088-3091).

Once autoantigens involved in particular autoimmune diseases have been identified, a variety of therapies have been proposed for inducing tolerance to such antigens. For example, International Patent Application PCT/US88/02139 discloses that oral or enteral administration of compounds derived from myelin basic protein may be effective in treating multiple sclerosis. Oral administration of autoantigens may result in immune tolerance, which is reportedly mediated by anergy, deletion or the generation of regulatory cells, depending on the dose of antigen administered. Oral administration of autoantigens has therefore been suggested as a therapy in human autoimmune and other inflammatory diseases (see for example Komagata Y, and Weiner H L., Oral tolerance, Rev Immunogenet 2000;2(1):61-73; and, Chaillous L. et al., 2000, “Oral insulin administration and residual beta-cell function in recent-onset type 1 diabetes: a multicentre randomised controlled trial” Lancet 12;356(9229):545-9). Screening tests for type 1 diabetes that are available involve the detection of predictive antibodies such as islet cell autoantibodies (ICAs), insulin autoantibodies (IAAs other antibodies directed at beta cell proteins or involve tests of beta cell dysfunction, such as first-phase insulin release. There remains a need for earlier and better diagnosis and therapy for patients who will develop or who suffer from type 1 diabetes.

### SUMMARY OF THE INVENTION

In various alternative aspects, the invention provides compounds and methods relating to the diagnosis, therapy, prevention, or prophylaxis of type 1 diabetes.

In one aspect, the invention provides diagnostic methods that may be used to provide information about a disease state in a subject. In some embodiments, the invention provides diagnostic methods for providing information about a type 1 diabetes disease state in a human patient. In alternative aspects, the invention provides methods of modulating an immune response, for example in a human patient. Such methods may involve contacting a sample, such as a sample comprising a T lymphocyte from the patient, with a diagnostic or therapeutic compound comprising a diagnostic or therapeutic epitope of Formula I. The diagnostic or therapeutic compound may bind to the T lymphocyte with an affinity that is at least as great as the affinity when the diagnostic or therapeutic epitope is KLQVFLIVL (HTV-1), KLNERLAKL (HTV-5) or an alternative human beta-cell-protein-leader-sequence-derived peptide sequence such as a human IAPP-leader-sequence derived peptide. In alternative embodiments, particularly for use in animal models of disease, the affinity may be at least as great as the affinity when the diagnostic epitope is KLPAVLLIL (mTV-1) or an alternative animal beta-cell-protein-leader-sequence-derived peptide sequence such as an animal (murine) IAPP-leader-sequence derived peptide.

Compounds of Formula (I) may for example be derived from other epitopes of the invention through substitution, or through random synthesis, and may have the following structure:
Z₁-X₋₁-X₁-X₂-X₃-X₄-X₅-X₆-X₇-X₈-X₉-X₊₁-Z₂; (I)
Wherein, particularly for use in humans,
X₋₁ at each occurrence is independently selected from any amino acid or analogue thereof or is absent;
X₁ may for example be Lys (K) or a hydrophilic amino acid selected from the group consisting of T, H, E, Q, N, R, S or K; or an amino acid having a similar hydrophilicity value, such as Lys (+3.0), Arg (+3.0), Asp (+3.0) or Glu (+3.0); a basic amino acid; any amino acid or analogues thereof;
X₂ may for example be Leu (L), may be Met (M), or may be selected from Leu, Met, Ile, Phe, Ala, Gly, Val, Trp; or any amino acid or analogues thereof;
X₃ may for example be any amino acid; may be Q; may be N; may be a hydrophilic amino acid selected from T, H, E, Q, N, R, S or K; may be an amino acid having a hydrophilicity value of about 0.2, such as Gln, Asn, Ser, or Gly; may be an amino acid having an hydropathic index of about −3.5 such as Glu, Gln, Asp, Asn, or optionally Lys; a polar amino acid; or analogues thereof;
X₄ may for example be any amino acid; may be V; may be E; an apolar amino acid; an acid amino acid; or analogues thereof;
X₅ may for example be any amino acid; may be F; may be R; an aromatic amino acid; a basic amino acid; or analogues thereof;

In alternatvie embodiments one of X4 and X5 is a hydrophobic amino acid, such as F or V and the other of X4 and X5 is a hydrophilic amino acid, such as E or R;
X₆ may for example be L; or may be any amino acid; or may be a hydrophobic amino acid such as Val or Phe; an aliphatic amino acid; or analogues thereof;
X₇ may for example be I; may be A; may be a hydrophobic amino acid selected from Ile, Val, Leu, Phe, Cys, Met, or Ala; an apolar amino acid; an aliphatic amino acid; or may be any amino acid; or analogues thereof;
X₈ may for example be V; may be K; may be any amino acid; an apolar amino acid; an aliphatic amino acid; a basic amino acid; or analogues thereof;
X₉ may for example be L; may be V; may be Leu, Ile, or Val; or any amino acid or analogues thereof
X₊₁ may for example be any amino acid or analogues thereof or is absent;
Z₁ may be H₂N—, RHN— or, RRN—;
Z₂ may for example be —C(O)OH, —C(O)R, —C(O)OR, —C(O)NHR, —C(O)NRR;
R at each occurrence may for example be independently selected from (C₁-C₆) alkyl, (C₁-C₆) alkenyl, (C₁-C₆) alkynyl, substituted (C₁-C₆) alkyl, substituted (C₁-C₆) alkenyl, or substituted (C₁-C₆) alkynyl;
wherein “−” is a covalent linkage;
and wherein in some embodiments X₋₁ and X₊1 cannot both be present (so that the length of the peptide is a maximum of 10 subunits).

In alternative embodiments, particularly for use in animals:
X₋₁ at each occurrence is independently selected from any amino acid or is absent;
X₁ may for example be Lys (K) or a hydrophilic amino acid selected from the group consisting of T, H, E, Q, N, R, S or K; or an amino acid having a similar hydrophilicity value, such as Lys (+3.0), Arg (+3.0), Asp (+3.0) or Glu (+3.0); or analogues thereof;
X₂ may for example be Leu (L), may be Met (M), or may be selected from Leu, Met, Ile, Phe, Ala, Gly, Val, Trp or analogues thereof;
X₃ may for example be any amino acid; may be P; may be a hydrophobic amino acid selected from G, A, F, V, L, I, P, M or W; may be an amino acid having a hydropathic index of about −1.6 such as Tyr (−1.3) or Pro (−1.6); may be an amino acid having a may be an amino acid having a hydropathic index of about −0.5 such as Pro (−0.5), Thr (−0.4), Ala (−0.5) or His (−0.5); or analogues thereof;
X₄ may for example be any amino acid; may be A; or analogues thereof;
X₅ may for example be any amino acid; may be V; or analogues thereof;
X₆ may for example be L; or may be any amino acid; or may be a hydrophobic amino acid such as Val or Phe; or analogues thereof;
X₇ may for example be L; may be a hydrophobic amino acid selected from Ile, Val, Leu, Phe, Cys, Met, or Ala; or may be any amino acid; or analogues thereof
X₈ may for example be I; may be any amino acid; or analogues thereof;
X₉ may for example be L; may be V; may be Leu, Ile, or Val; or analogues thereof
X₊₁ may for example be any amino acid or is absent; or analogues thereof;
Z₁ may be H₂N—, RHN— or, RRN—;
Z₂ may for example be —C(O)OH, —C(O)R, —C(O)OR, —C(O)NHR, —C(O)NRR;
R at each occurrence may for example be independently selected from (C₁-C₆) alkyl, (C₁-C₆) alkenyl, (C₁-C₆) alkynyl, substituted (C₁-C₆) alkyl, substituted (C₁-C₆) alkenyl, or substituted (C₁-C₆) alkynyl;
wherein “−” is a covalent linkage;
and wherein in some embodiments X₋₁ and X₊₁ cannot both be present (so that the length of the peptide is a maximum of 10 subunits).

In alternative embodiments, therapeutic and diagnostic methods of the invention may be carried out in vivo or in vitro. Diagnostic and therapeutic methods may also be repeating over a time course, for example a diagnostic method may include collecting first and second samples from the patient at a first time-point and a second time-point respectivley, to detect an increase, a decrease, or no change in a cytotoxic T lymphocyte response between the first time-point and the second time-point.

In one aspect, the invention provides methods of treating an animal, such as a NOD mouse, to provide an animal model of type 1 diabetes, and animals produced by such methods, wherein an animal is treated with an immunogenic compound having a immunogenic epitope of Formula I.

In alternative aspects, the invention provides methods for isolating T lymphocytes, such as cytotoxic T lymphocytes, from a subject such as a human patient. For example, T lymphocytes may be isolated based on the binding of the lymphcyte to the diagnostic or therapeutic compounds of the invention. TCRs or T lymphocytes isolated in this way may in turn be used to provide tolerizing vaccines of the invention, in which such TCRs or T cells are used in immunogenic compositions administered to a subject.

Compounds and epitopes of the invention may for example be provided in combination with other compounds that together define an immunogenic entity or an epitope recognized by a ligand. For example, compounds of the invention may be provided in combination with a major histocompatibility complex molecule, such as a class I molecule, such as HLA-A*0201 or portions thereof or multimers constructed from such molecules.

### BRIEF DESCRIPTION OF THE DRAWINGS AND SEQUENCE LISTING

FIG. 1 is a schematic representation of a photograph of the results of an interferon-gamma ELISPOT assay, showing that murine autoreactive T cells recognise TV1 and the positive control peptide, NRP-V7, but not the negative control peptide, TUM. Other peptides tested in the assay include INSL, TV2, and TV3. The scoring of the results shows increasing reactivity denoted with the symbols: −, +, ++.

FIGS. 2A-H are flow cytometry histograms showing that a significant percentage of antigen presenting cells that express the murine MHC class I molecule, H-2Kd, bind and present a TV peptide on the cell surface. In the absence of a peptide that binds to H-2Kd, there is a negligible proportion of stable H-2Kd on the cell surface. FIG. 2A shows a forward and side scatter plot indicating the population of antigen presenting cells. FIG. 2B shows a negative control (unstained) population of cells. FIG. 2C-H show the proportion of H-2Kd on the surface of the APC in the presence of TUM (66%), NRP-V7 (72%), INS-L (73%), TV1 (64%), TV2 (58%) and TV3 (70%).

FIGS. 3A-M are flow cytometry dot plots showing that autoreactive (tetramer-positive) T cells accumulate within the pancreatic islets of NOD mice as they age and develop clinical disease. FIG. 3A shows indicates the proportion of pancreatic islet CD8+ T cells (encircled in each upper right quadrant) also tetramer-positive for TUM, NRP-V7 and INS-L. FIG. 3M is a bar graph summarising the results of FIGS. 3A-L.

FIG. 4 is a graph showing the detection of autoreactive (tetramer-positive) T cells in peripheral blood from a single mouse. A single female NOD mouse was followed weekly for blood glucose (diamonds, black line) and blood tetramer-positive frequency (squares, grey line) from 9 to 18 weeks of age. The arrows correspond to the actual flow cytometry data illustrating tetramer negative (A) and tetramer positive (B) populations of T cells.

FIG. 5 is a bar graph showing that the autoreactive T cells in NOD pancreatic islets secrete interferon-gamma in response to the previously identified autoepitope NRP-V7, but minimally in response to the autoepitope INSL, and not to the peptide TUM.

FIG. 6 is a line graph showing glucose concentration prior to, during, and after onset of hyperglycaemia (diamonds, black line). The percentage of CD8 expressing cells that are NRP-V7 tetramer positive is also shown (squares, grey line). Pooled data from all hyperglycaemic mice were used, normalised to the time at which hyperglycaemia appears (Time 0). Y-axis is Serum Glucose [mM].

FIG. 7 is a line graph showing that autoreactive T cells appear in waves or in cyclical fashion prior to onset of hyperglycaemia. The percentage of mice that are diabetic (diamonds, black line) and the percentage of CD8 expressing cells that are NRP-V7 tetramer positive (squares, grey line) are shown.

FIG. 8 is a bar graph depicting the results of an interferon-gamma ELISPOT assay showing that human autoreactive T cells recognise the HLA-A*0201 restricted peptide epitopes HIV, FLU, and HTV1. MED refers to medium alone. Error bars represent standard deviation of the mean. The Y-axis is “Spot-forming units”.

FIG. 9 is a line graph showing the number of mice (Y axis) having diabetic onset over a time course of 10 weeks to 23 weeks following immunization beginning at 3 weeks of age with a peptide of the invention (MTV-1). NOD mice were immunised with various peptides: dark circles identify mice immunized with the negative control peptide TUM, white squares identify mice immunized with the positive control peptide NRP-A7, white circles identify mice immunized with the peptide MTV-1 (TV1).

FIG. 10 is a schematic representation of a photograph of results from an interferon gamma ELISPOT showing T cell reactivity against epitopes of the invention, HTV1 and HTV5, in a type 1 diabetic patient with residual beta cell function. The scoring of the results (with increasing reactivity denoted with the symbols: −, ++, +++) reflects the relative T cell reactivity against the peptides HCV (hepatitis C peptide), HTV1, HTV5 and the positive control PHA were used.

FIG. 11 is a histogram showing that the peptide HTV1 binds to the human MHC class I molecule HLA-A*0201.

FIG. 12 are flow cytometry dot plots showing CTL from pancreatic islets of NOD mice recognize MTV2. Islet cells were stained directly ex vivo or following in vitro culture with H-2Kd tetramers bearing MTV2 or TUM (negative control) to determine the proportion of CD8+tetramer+ T cells present.

### DETAILED DESCRIPTION OF THE INVENTION

“Type 1 diabetes,” as used herein, is a form of diabetes mellitus that is, at least in part, the result of an autoimmune response, i.e., an immune response in which the immune system of an animal reacts against the animal's own cells or tissues. In type 1 diabetes, the cellular immune system targets pancreatic beta cells. In general, as the diseases progresses, type 1 diabetes is characterised by relative or absolute insulin deficiency leading to uncontrolled carbohydrate metabolism.

When an antigen binds to its receptor, a T-cell receptor (TCR) or an antibody, only a relatively small part of that antigen typically contacts the receptor. That part of the antigen is called an epitope (also known as an antigenic determinant). In general, a peptide antigen recognized by a TCR is tightly bound to a groove in a MHC molecule. Accordingly, a T cell antigen must generally contain two distinct interaction sites; one interacts with the T cell receptor and is called the epitope; the second interacts with the MHC molecule and may be called the agretope. The size of a T cell epitope is generally on the order of 8-11 amino acids (9 may be best) for those epitopes which are associated with Class I molecules; 12-25 amino acids form an epitope associated with a Class II molecule. Epitopes recognized by antibodies may be made up of amino acids from different regions of an antigen, and 15-22 amino acids may actually contact the antibody binding site and make up the epitope. In alternative aspects of the invention, epitopes are provided that in one context may function as T cell epitopes associated with an MHC molecule, with or without an agretope, and in another context may function as a B cell epitope. Accordingly, epitopes of the invention are not restricted to sequential amino acids, nor are they necessarily linked to agretopes or other factets of an antigen that may in some circumstances be required for immune system recognition.

The surface of an antigen may have many potential B to T cell epitopes on it. Which ones the immune system of a particular animal will respond to will vary from species to species and from individual to individual within a species. The epitopes producing the strongest immune response in an animal are sometimes referred to as immunodominant epitopes. In the present application, a compound that is immunodominant for type 1 diabetes, or an type 1 diabetes “immunodominant compound” is a compound that has one or more epitopes, an “immunodominant epitope”, capable of being the target of an immune response, for example a cellular immune response, which mediates the pathology of type 1 diabetes. Accordingly, the use of the word “immunodominant” does not connote herein that a compound or epitope elicits the strongest immune response, merely that the compound or epitope elicits a clinically relevant immune response. In one aspect of the invention, immunodominant compounds or epitopes are characterised by the fact that, in a subject presenting with symptoms of type 1 diabetes a detectable proportion of immune system cells, for example cytotoxic T lymphocytes, will recognize the compound or epitope when the compound or epitope is presented in an appropriate context, such as within the binding groove of an MHC class I molecule or with an adjuvant. Such compounds can include polypeptides that are targets of the immune system in type 1 diabetes, and peptide analogues thereof (for example, organic compounds that mimic or antagonise cytotoxic T lymphocyte responses, T cell receptor-binding properties, MHC molecule-binding properties, etc. of polypeptides that are targets of the immune system in type 1 diabetes). In some aspects of the invention, compounds that are immunodominant also include compounds that may be used to decrease, stop, tolerise, neutralise or inhibit an immune response, such as compounds that may be used in a tolerising vaccine to ameliorate a cytotoxic T lymphocyte response in type 1 diabetes. Further discussion of immunodominant compounds according to the present invention is provided in the detailed description of the invention, herein, as well as in documents incorporated herein by reference.

An “IAPP leader peptide” is a peptide derived from the leader sequence (the sequence that is initially cleaved during proteolytic processing of the IAPP polypeptide) of a preproIAPP.

A “sample” can be any organ or tissue isolated from a subject, such as a sample isolated from a mammal having cytotoxic T lymphocytes. For example, a sample can include, without limitation, pancreatic tissue, pancreatic islet cells (e.g., beta cells), peripheral blood, etc., isolated from a mammal with type 1 diabetes, e.g., a diabetic human or a NOD mouse. A sample can also include cultured cells or cell lines.

A “cytotoxic T lymphocyte” or “CTL” is an immune system cell that recognises epitopes presented by class I MHC molecules through its TCR. A CTL will generally express the CD8 antigen on its cell surface. A “cytotoxic T lymphocyte response” or a “CTL response” occurs when a CTL encounters an antigen and responds, for example, by secreting cytokines such as (but not exclusively) gamma interferon (IFN-gamma). A CTL is “autoreactive” when it initiates an abnormal response and recognises and destroys “self” (native) antigens. A CTL is “type 1 diabetes autoreactive,” as used herein, when it recognises and destroys self pancreatic beta cells. A compound according to the present invention modulates a type 1 diabetes autoreactive cytotoxic T lymphocyte response if it affects, directly or indirectly, any event in the pathway leading to the recognition and destruction of self pancreatic beta cells by CTL. In various aspects, the invention may involve assaying for a CTL response, which may for example include the step of measuring any detectable modulation in T lymphocyte proliferation or activation in response to a challenge, for example in response to conditions that are a normally stimulatory. “Deleting, tolerising, or neutralising” an immune response means down-regulating the immune response by one or more measures of immune system activity. For example, tolerising a CTL response generally involves reducing the titre of active CTLs.

By “modulates” is meant changes, by either increase or decrease.

An “asymptomatic” mammal is a mammal (e.g., human, mouse, rat, pig, dog, monkey) that does not show any overt clinical symptoms of type 1 diabetes, such as high blood glucose, weakness, weight loss, vision problems, excessive hunger and thirst.

An “antigen presenting cell” or “APC” is any cell that carries antigen, bound to a major histocompatibility class I molecule, on its cell surface and presents the antigen in this context to a T cell. An antigen presenting cell can include, without limitation, an endothelial cell, a dendritic cell, a spleen cell, a macrophage, or any cell line, such as RMAS-Kd or P815. Antigen presenting cells are generally incubated with a peptide, (usually a nonapeptide, although peptides in the range of eight to ten amino acids can be used), that enables direct binding of the peptide to the MHC molecule of the APC. An antigen presenting cell can exogenously acquire a compound by being incubated in the presence of the compound. Larger molecules, such as larger peptides or nucleic acid molecules encoding larger peptides, can be introduced into an APC (by transfection, electroporation, liposome fusion, osmotic shock, etc.), such that they are processed endogenously and peptides of the appropriate size are expressed on the cell surface of the APC.

A “major histocompatibility complex molecule” or “MHC molecule” is a cell surface glycoprotein that is involved in mediating the immune response in mammals by presenting an antigenic peptide to a specific T cell receptor. In mammals, major histocompatibility complex molecules can be class I or class II molecules. Class I MHC molecules “present” endogenous (protein made in the cell) or exogenous (protein acquired from outside the cell) peptides to a specific T cell receptor for recognition by the T cell, and in the case of an autoimmune response, present self-peptides to the T cell receptor. Class I MHC molecules include HLA-A, B and C molecules in humans, H2-D and K in mice, RLA in rabbits, RT 1 in rats, DLA in dogs, SLA in pigs, etc. Common HLA molecules in humans include HLA*0201, HLA-A*11, A*03, HLA-B*08, B*07, B*35.______. Common H2 molecules in inbred laboratory mice include H2-Kd, H-2Kb, H2-Dd, H2-Db______. A compound according to the present invention can be provided “in combination with a major histocompatibility molecule” if the compound is present in a sample containing a MHC molecule, or is present in a sample containing an antigen presenting cell. A MHC molecule can be in a multimer, for example, a tetramer, form. A “major histocompatibility complex class I binding motif” includes a Leu, Met, Ile, Phe, Ala, Gly, Val, or Trp residue five (5) amino acids N-terminal to a Val, Leu, or Ile residue.

A “test compound” is any chemical compound, be it naturally-occurring or artificially-derived. Test compounds may include, without limitation, peptides, polypeptides, synthesised organic molecules, naturally occurring organic molecules, and nucleic acid molecules. A test compound can “compete” with a known compound by, for example, interfering with binding of the known compound to a MHC molecule; interfering with binding of the known compound/MHC molecule complex to the cognate T cell receptor; or by interfering with any CTL response induced by the known compound.

A “pancreatic beta cell leader peptide” is any polypeptide that is naturally found in the islet beta cells of the pancreas of a mammal, and that includes a signal sequence or leader sequence that is normally cleaved off during proteolytic processing of the polypeptide. A pancreatic beta cell leader peptide can also include a fragment of a polypeptide that is naturally found in beta cells, as long as the fragment includes the signal or leader sequence. A pancreatic beta cell leader peptide is “expressed” in a cell if it is detectable in the cell, or in a lysate of the cell, by any method known in the art, such as by Northern or Southern blot, immunoprecipitation, immunoblot, etc. A pancreatic beta cell leader peptide is “preferentially expressed” in a pancreatic beta cell if it is expressed at a level that is at least 20% greater than in another cell type, or is expressed at a level that is 50% greater than in another cell type, or is expressed at a level that is 90% or more than 100% greater than in another cell type.

A compound is “substantially pure” when it is separated from the components that naturally accompany it. Typically, a compound is substantially pure when it is at least 60%, more generally 75% or over 90%, by weight, of the total material in a sample. Thus, for example, a polypeptide that is chemically synthesised or produced by recombinant technology will be generally be substantially free from its naturally associated components. A nucleic acid molecule is substantially pure when it is not immediately contiguous with (i.e., covalently linked to) the coding sequences with which it is normally contiguous in the naturally occurring genome of the organism from which the DNA of the invention is derived. A substantially pure compound can be obtained, for example, by extraction from a natural source; by expression of a recombinant nucleic acid molecule encoding a polypeptide compound; or by chemical synthesis. Purity can be measured using any appropriate method such as column chromatography, gel electrophoresis, HPLC, etc.

The term “alkyl” refers to the radical of saturated aliphatic groups, including straight chain alkyl groups, branched-chain alkyl groups, cycloalkyl (alicyclic) groups, alkyl substituted cycloalkyl groups, and cycloalkyl substituted alkyl groups. Typical alkyl groups include, but are not limited to, methyl, ethyl, propyl, isopropyl, butyl, isobutyl, t-butyl, pentyl, isopentyl, hexyl, etc. The alkyl groups can be (C₁-C₆) alkyl, or (C₁-C₃) alkyl. A “substituted alkyl” has substituents replacing a hydrogen on one or more carbons of the hydrocarbon backbone. Such substituents can include, for example, halogen, hydroxyl, carbonyl (such as carboxyl, ketones (including alkylcarbonyl and arylcarbonyl groups), and esters (including alkyloxycarbonyl and aryloxycarbonyl groups)), thiocarbonyl, acyloxy, alkoxyl, phosphoryl, phosphonate, phosphinate, amino, acylamino, amido, amidine, imino, cyano, nitro, azido, sulfhydryl, alkylthio, sulfate, sulfonate, sulfamoyl, sulfonamido, heterocyclyl, aralkyl, or an aromatic or heteroaromatic moiety. The moieties substituted on the hydrocarbon chain can themselves be substituted, if appropriate. For instance, the substituents of a substituted alkyl may include substituted and unsubstituted forms of aminos, azidos, iminos, amidos, phosphoryls (including phosphonates and phosphinates), sulfonyls (including sulfates, sulfonamidos, sulfamoyls and sulfonates), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The terms “alkenyl” and “alkynyl” refer to unsaturated aliphatic groups analogous in length and possible substitution to the alkyls described above, but that contain at least one double or triple bond respectively. An “alkenyl” is an unsaturated branched, straight chain, or cyclic hydrocarbon radical with at least one carbon-carbon double bond. The radical can be in either the cis or trans conformation about the double bond(s). Typical alkenyl groups include, but are not limited to, ethenyl, propenyl, isopropenyl, butenyl, isobutenyl, tert-butenyl, pentenyl, hexenyl, etc. An “alkynyl” is an unsaturated branched, straight chain, or cyclic hydrocarbon radical with at least one carbon-carbon triple bond. Typical alkynyl groups include, but are not limited to, ethynyl, propynyl, butynyl, isobutynyl, pentynyl, hexynyl, etc.

A “substantially identical” sequence is an amino acid or nucleotide sequence that differs from a reference sequence only by one or more conservative substitutions, as discussed herein, or by one or more non-conservative substitutions, deletion, or insertions located at positions of the sequence that do not destroy the biological function of the test compound. Such a sequence can be at least 60% or 75%, or more generally at least 80%, 85%, 90%, or 95%, or as much as 99% identical at the amino acid or nucleotide level to the sequence used for comparison. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine). Examples of useful software include the programs, Pile-up and PrettyBox. Such software matches similar sequences by assigning degrees of homology to various substitutions, deletions, substitutions, and other modifications.

A “T cell receptor” or “TCR” is the antigen recognising receptor on the surface of T cells or T lymphocytes. A TCR generally binds antigens, such as peptides, in association with a specific MHC molecule, an MHC-peptide complex, which may lead to activation of the T cell. A TCR, as used herein, includes naturally occurring TCRs as well as synthetic variant of the TCR such as solubilized TCRs and single chain TCRs (see for example Engel et al. 1992. High-efficiency expresion and solubilization of functional T cell antigen receptor heterodimers. Science 256(5061):1318-21).

A “peptide” or “polypeptide” is any chain of two or more amino acids, including naturally occurring or non-naturally occurring amino acids or amino acid analogues, regardless of post-translational modification (e.g., glycosylation or phosphorylation). An “amino acid sequence”, “polypeptide”, “peptide” or “protein” of the invention may include peptides or proteins that have abnormal linkages, cross links and end caps, non-peptidyl bonds or alternative modifying groups. Such modified peptides are also within the scope of the invention. The term “modifying group” is intended to include structures that are directly attached to the peptidic structure (e.g., by covalent coupling), as well as those that are indirectly attached to the peptidic structure (e.g., by a stable non-covalent association or by covalent coupling to additional amino acid residues, or mimetics, analogues or derivatives thereof, which may flank the core peptidic structure). For example, the modifying group can be coupled to the amino-terminus or carboxy-terminus of a peptidic structure, or to a peptidic or peptidomimetic region flanking the core domain. Alternatively, the modifying group can be coupled to a side chain of at least one amino acid residue of a peptidic structure, or to a peptidic or peptidomimetic region flanking the core domain (e.g., through the epsilon amino group of a lysyl residue(s), through the carboxyl group of an aspartic acid residue(s) or a glutamic acid residue(s), through a hydroxy group of a tyrosyl residue(s), a serine residue(s) or a threonine residue(s) or other suitable reactive group on an amino acid side chain). Modifying groups covalently coupled to the peptidic structure can be attached by means and using methods well known in the art for linking chemical structures, including, for example, amide, alkylamino, carbamate or urea bonds.

As used herein, the term “amino acids” means those L-amino acids commonly found in naturally occurring proteins, D-amino acids and such amino acids when they have been modified. Accordingly, amino acids of the invention may include, for example: 2-Aminoadipic acid; 3-Aminoadipic acid; beta-Alanine; beta-Aminopropionic acid; 2-Aminobutyric acid; 4-Aminobutyric acid; piperidinic acid; 6-Aminocaproic acid; 2-Aminoheptanoic acid; 2-Aminoisobutyric acid; 3-Aminoisobutyric acid; 2-Aminopimelic acid; 2,4 Diaminobutyric acid; Desmosine; 2,2′-Diaminopimelic acid; 2,3-Diaminopropionic acid; N-Ethylglycine; N-Ethylasparagine; Hydroxylysine; allo-Hydroxylysine; 3-Hydroxyproline; 4-Hydroxyproline; Isodesmosine; allo-Isoleucine; N-Methylglycine; sarcosine; N-Methylisoleucine; 6-N-methyllysine; N-Methylvaline; Norvaline; Norleucine; and Ornithine.

A “nucleic acid molecule” is any chain of two or more nucleotides including naturally occurring or non-naturally occurring nucleotides or nucleotide analogues.

An antibody “specifically binds” an antigen when it recognises and binds the antigen, but does not substantially recognise and bind other molecules in a sample, having for example an affinity for the antigen which is 10, 100, 1000 or 10000 times greater than the affinity of the antibody for another reference molecule in a sample.

Idiotypic epitopes are the parts of an immunoglobulin molecule that are present in the variable region, which are generally unique to that specific immunoglobulin molecule and are typically involved in binding to its antigen. An immune response may be directed against the idiotypic epitope of an antibody, and antibodies may be generated which can bind to that antibody, called anti-idiotype antibodies. Anti-idiotype antibodies may recognize the antigen binding site or idiotypic epitope of an immunoglobulin and may therefore resemble the original antigen. Accordingly, one aspect of the invention provides antibodies having idiotypic epitopes that bind to the epitopes or antigens of the invention. In another aspect, the invention provides anti-idiotype antibodies that can bind to such idiotypic epitopes. Such anti-idotype antibodies may be used in aspects of the invention as substitutes for the epitopes of the invention. For example, anti-iotype antibodies may be conjugated to labels or chemotherapeutic agents to modulate the T cell response to the epitopes of the inventon.

Immunogenicity is the ability to induce either a humoral or a cell-mediated immune response. Antigenicity is the ability to combine specifically with the final products of the immune response (generally an antibody or a TCR). If a molecule is immunogenic, it is also antigenic; however, a molecule can be antigenic without being immunogenic (for example haptens can bind to antibodies but cannot, on their own, elicit an immune response). Accordingly, antigenic compounds of the invention are not necessarily immunogenic.

Tolerogenicity is the ability to induce specific immune nonresponsiveness or tolerance. Tolerance is used to describe the specific unresponsiveness of an immune system to an antigen. Although tolerance occurs in both T-cell and B-cell populations, tolerance can generally be induced more easily and more quickly in T cells, and this T cell tolerance may last for months. T cell tolerance may for example be induced by negative selection during T cell maturation in the thymus, or a mature T cell may be functionally inactivated by a process known as clonal anergy. Accordingly, tolerizing antigens of the invention may induce unresponsiveness in one or more immune system cell type, and this tolerance may persist for variable periods of time. A tolerizing dose of an epitope or compound of the invention is a dose administered in an amount and over a period of time that is sufficient to induce tolerance in the subject. Other features and advantages of the invention will be apparent from the following description of the invention and drawings, the attached drawings, and from the claims. In addition, the teachings of all patents and publications cited in this specification are specifically incorporated by reference in their entirety as if each were explicitly incorporated herein.

The invention provides, in part, compounds that are immunodominant for type 1 diabetes, and methods for using such compounds. The compounds of the invention include IAPP (islet amyloid polypeptide) precursor peptides and analogues thereof.

IAPP, also known as amylin, is a secreted protein that is primarily expressed in beta cells (Hoppener, J. et al., 1992). Human IAPP is first synthesised in the beta cell as an 89 amino acid precursor molecule (GenBank accession nos. X68830 S52418; SWISS-PROT: P10997) called preproIAPP (of SEQ ID No: MGILK LQVFL IVLSV ALNHL KATPI ESHQV EKRKC NTATC ATQRL ANFLV HSSNN FGAIL SSTNV GSNTY GKRNA VEVLK REPLN YLPL). A short “leader” sequence is immediately cleaved in the endoplasmic reticulum of the beta cell to produce the 67 amino acid proIAPP. ProIAPP is subsequently cleaved in beta cell secretory granules to produce the 37 amino acid mature IAPP, the major secreted form. Murine IAPP (GenBank accession no. NM_{—}010491, version NM_{—}010491.1 GI:6754271; P12968) is initially synthesised as a 93 amino acid precursor (of SEQ ID No?: MMCIS KLPAV LLILS VALNH LRATP VRSGS NPQMD KRKCN TATCA TQRLA NFLVR SSNNL GPVLP PTNVG SNTYG KRNAA GDPNR ESLDF LLV) that undergoes proteolytic processing events, similar to human IAPP, to form a 70 amino acid proIAPP, and subsequently, a 37 amino acid mature IAPP (see Ekawa, K. et al., 1997).

In one aspect, the invention provides IAPP precursor peptides of the invention are type 1 diabetes immunodominant epitopes, and include the peptides KLQVFLIVL, KLPAVLLIL, KLNERLAKL, QVFLIVLSV, GILKLQVFL, FLIVLSVAL, or VLSVALNHL (SEQ ID NOs:______).

Detection of Cytotoxic T Lymphocytes

Antigen-specific CTLs can be detected using a wide variety of assays, including immunospot (e.g., ELISPOT) assays, MHC class I tetramer assays, or other assays, as described herein or as known to a person skilled in the art. In one aspect of the invention, assays can be performed using the compounds of the invention, for example, IAPP precursor peptides, to detect antigen-specific CTLs.

ELISPOT assays are a powerful tool for the detection and analysis of cells secreting a particular protein, and can be used to determine the effectiveness of various peptides or other compounds as epitopes for diabetes by measuring gamma interferon or other cytokine secretion by activated CTLs.

MHC tetramers are complexes of four MHC molecules, in combination with a specific peptide and a fluorochrome. Such complexes are capable of binding to a distinct subset of TCRs. Thus, MHC tetramers enable detection and quantification of T cells specific for a single peptide, regardless of functionality. MHC class I tetramers, such as those disclosed in U.S. Pat. No. 5,635,363, can be used for detection of autoreactive cytotoxic T lymphocytes (CTL) indicative of type 1 diabetes.

Diagnosis or Prediction of Type 1 Diabetes

Patients with type 1 diabetes generally have an increasing frequency of CTL that recognise autoantigens. Such autoreactive CTL may be detected, for example, both in pancreatic islet cell tissues and in peripheral blood. Since the generation of autoreactive CTL is thought to precede the development of autoantibodies and other indicia of the clinical symptoms of diabetes, detection of autoreactive CTL using compounds according to the present invention may in some cases enable more sensitive and specific diagnosis or prediction of type 1 diabetes.

In some embodiments, compounds according to the present invention, for example, IAPP precursor peptides, can be used to assay CTL responses, and thus detect or diagnose type 1 diabetes autoreactive CTL. The assays can also be used to quantify both the absolute number and the proportion of autoreactive CTL present in a sample, such as a peripheral blood sample, in both pre-diabetic (pre-clinical) subjects and diabetic patients using peptide-specific CTL detection assays. In some embodiments, both the severity and course of diabetes may be predicted and followed using such assays. For example, the human MHC class I molecule HLA-A*0201 can be used in combination with an IAPP precursor peptide to detect autoreactive CTL present in a peripheral blood sample of a pre-diabetic subject.

The compounds and methods of the invention can therefore be used to test a subject who is suspected of having type 1 diabetes, or is suspected to be at risk for type 1 diabetes. Since close family members of type 1 diabetic patients have a greater probability of developing type 1 diabetes, compared with the general population, it may be advisable to test such family members for the presence of type 1 diabetes autoreactive CTL. Additionally or alternatively, if a subject has a family history of type 1 diabetes, diagnostic, predictive, or other tests according to the invention may be warranted. The tests can be carried out at intervals, e.g., annually, to continue to monitor a subject who is suspected of having type 1 diabetes, or is suspected to be at risk for type 1 diabetes.

In some embodiments, an advantage of the invention is that it is possible to carry out tests, such as diagnostic or predictive tests, in a relatively non-invasive manner by, for example, assaying in vitro CTL present in peripheral blood samples. Alternatively, in some embodiments, the compounds of the invention can also be used in vivo in combination with, for example, imaging techniques or other in vivo detection methods for detecting CTLs labelled by binding with compounds of the invention.

Monitoring the Degree of Progression or Response to Therapy of Type 1 Diabetes

The loss of pancreatic beta cells is a gradual process. The invention can be used to monitor the rate of loss of pancreatic beta cells in a subject with type 1 diabetes by detecting or quantifying type 1 diabetes autoreactive CTL at different points in time, to get an indication of the severity of the disease in the subject. The results of such monitoring can be used to assess how to manage the disease in the particular subject, by for example, determining what therapy should be used and how aggressively to pursue different treatment alternatives.

The invention can also be used to monitor the response of a subject who is receiving therapy for type 1 diabetes, by determining if the therapy is having any effect on type 1 diabetes autoreactive CTL in the subject (e.g., if the therapy is reducing the number of autoreactive CTL, or preventing the proliferation of autoreactive CTL). Thus, compounds and methods according to the present invention can be used to observe response to therapy in patients with type 1 diabetes by, for example, quantifying the number and/or proportion of autoreactive CTL in peripheral blood before, during, or after treatment.

The monitoring should be carried out over at least two time points to get an indication of any difference in type 1 autoreactive CTLs in the elapsed time period. The monitoring can also be carried out at multiple time periods over a subject's life, or over any period deemed appropriate.

The monitoring can be carried out by, for example, assaying CTL present in peripheral blood in vitro, or by using in vivo imaging or other techniques. For example, following a medical procedure or therapy, such as islet cell transplantation, assays of the invention may be carried out to assess an immune reaction against an epitope of the invention, for example to monitor autoreactive T cell counts.

Therapy or Prophylaxis of Type 1 Diabetes

Compounds according to the present invention, for example, peptide or non-peptide analogues of the peptides described herein, or identified according to the methods herein, can be used to modify type 1 diabetes autoreactive CTLs, by for example, deleting, tolerising, neutralising, or otherwise rendering ineffectual CTL reactive to them, thus preserving islet beta cell function. Such compounds can be used to treat type 1 diabetes, and can be administered to individuals at risk for developing type 1 diabetes, newly diagnosed with type 1 diabetes, with longstanding type 1 diabetes, or following transplantation of the pancreas or pancreatic islets.

Compounds useful for therapy or prophylaxis can include peptides or peptide analogues that have been modified by, for example, amino acid substitution, insertion, or deletion, relative to native IAPP precursor peptides, for example, KLQVFLIVL, KLPAVLLIL, KLNERLAKL, QVFLIVLSV, GILKLQVFL, FLIVLSVAL, or VLSVALNHL (SEQ ID NOs:______), such that the compounds bind effectively to an MHC class I molecule, but have reduced or enhanced ability to stimulate type 1 diabetes autoreactive CTL. The compounds therefore prevent CTL activation and proliferation, thus protecting pancreatic beta cells from targeting and destruction. These analogues compete with native LAPP precursor peptides, for binding to the MHC class I molecule. In general, the modifications will be conservative changes.

The compounds are aimed at inhibiting or suppressing antigen responses specific to type 1 diabetes patients, without affecting other, normal immune responses. In this context, it should be noted that the present invention provides compounds and methods related to the modulation of deleterious T lymphocytes, i.e., those T lymphocytes that promote a type 1 diabetes autoimmune attack. An important aspect of the present invention is the ability to modulate CTL in a specific manner, without significantly affecting normal immune function.

Immunological tolerance refers generally to a selective inability of immune system cells to respond to an antigen. Antigens in this sense includes epitopes that, in the absence of tolerance, cause an immune response, such as the destruction of pancreatic beta cells. Tolerance may be distinguished from immune suppression, both mechanistically and clinically, although suppressor cells can mediate tolerance. For example, tolerance may be considered to be antigen-specific and to persist after exposure to the tolerising agent has ceased. Tolerance is understood to function against both foreign and self antigens, and is thought to be maintained by active or passive processes which result from cell inactivation, altered cellular function, or cell death. Tolerance is understood to be induced centrally (in the thymus) and peripherally, in a number of ways, including by blocking the activation of T cells by antigen presenting cells (for example, by blocking the interaction of the TCR with peptides presented by a MHC molecule), or by blocking the production of costimulatory factors such as cytokines or by preventing costimulatory signalling.

In some embodiments, compounds according to the invention can be administered as tolerising vaccines. A tolerising vaccine, as used herein, is a pharmaceutically acceptable composition that includes at least one compound that, when administered according to an appropriate immunisation schedule, can induce immunological tolerance. For example, tolerizing vaccines of the invention may act to diminish reactivity of a T cell to an antigen. In some embodiments, tolerizing vaccines of the invention may be administered to tolerize type 1 diabetes autoreactive CTL responses, so as to prevent or treat type 1 diabetes. Thus, in some embodiments, a tolerising vaccine according to the present invention decreases, stops, or otherwise neutralises or inhibits a type 1 diabetes autoreactive cytotoxic T lymphocyte response. Tolerisation can be tested in animal models of type 1 diabetes, such as the NOD mouse, by adoptive transfer of autoantibody or autoreactive T cells.

A compound according to the present invention, for example an IAPP leader peptide as described herein, can be made tolerogenic by, for example, conjugation with a tolerogenic polymer (e.g., monomethoxypolyethylene glycol, polyvinyl alcohol, or any other compound that, when coupled to an antigen, causes loss of antigenicity of the antigen). Alternatively, a compound according to the present invention can be tolerogenic in itself, by administration of different doses to induce tolerance. For alternative methods of making and administering tolerizing vaccines, see U.S. Pat. Nos. 6,036,957, 6,039,947, 6,355,238, 4,838,852, (all of which are hereby incorporated by reference).

Effective tolerising doses can include doses of a tolerising vaccine that are capable of alleviating a type 1 diabetes autoimmune response by a mammal. A first tolerising dose can include an amount of a tolerogenic vaccine that causes a minimal autoimmune response or type 1 diabetes autoreactive CTL response when administered to a mammal having or at risk for type 1 diabetes. A second tolerising dose can include a greater amount of the same tolerising vaccine than the first dose. Effective tolerising doses can include increasing concentrations of the tolerising vaccine necessary to tolerise an mammal such that the mammal does not progress to the insulitis and the destruction of pancreatic beta cells. A single dose of a tolerogenic vaccine can be from 0.01 microgram to about 1,000 mg, or can be from 0.1 microgram to about 100 mg, or from about 1.0 microgram to about 10 mg, depending on the subject. For example, administration of both high- and low-dose antigen may in some embodiments induce immunological tolerance, where repeated antigen challenge diminishes immune response to subsequent systemic administration of antigen.

In one aspect, the invention provides methods for isolating autoreactive T cells, such as T cells that have a selective affinity for the peptides of the invention. Such isloated T-cells may be used in other aspects of the invention in order to vaccinate a subject against such T-cells, and thereby treat type 1 diabtes. For example, photopheresis may be used to treat autoreactive T-cells isolated from type 1 diabetes patients using the epitopes of the invention, and the treated autoreactive T-cells may then be administered to the patient, or a different patient, in order to reduce or eliminate autoreactive T-cell lineages.

In accordance with one aspect of the invention, peptides derived from IAPP have been identified as autoantigens in type 1 diabetes. Accordingly, one aspect of the invention provides methods for inducting tolerance to epitopes of the invention, for example epitopes on IAPP-derived-peptides. Such methods may for example include administration to subjects of proteins having the epitopes of the invention, such as IAPP or fragments thereof including the IAPP leader peptide. Oral administration of proteins comprising epitopes of the invention may for example be used to induce tolerance in some embodiments.

A tolerising vaccine of the invention can be administered by any appropriate route, for example, by oral or mucosal administration. Other routes of non-oral tolerance induction, such as by nasal or respiratory routes, can also be used and have the advantage that enzymatic degradation in the gastrointestinal tract can be elimination, and therefore, lower doses of antigen may be required.

Alternative methods are available for inhibiting a selected T cell response. For example, U.S. Pat. No. 6,083,503 discloses methods for using interleukin-2 to stimulate T cell death in the treatment of autoimmune disease. According, in some embodiments, compounds comprising epitopes of the invention may be administered to a subject, to cause T cells recognizing the epitopes to express IL-2, followed by administration of IL-2 in a dose effective to reduce the level of such T cells.

In some embodiments, compounds or epitopes of the invention may be administered to a patient through gene therapy. For example, vectors such as adeno-associated virus (AAV) may be used as a vehicle for therapeutic gene delivery to transform pancreatic islet cells or antigen presenting cells for transplantation (see for example Kapturozak and Atkinson, 2001; and, Yamaoka T., 2001).

Compounds

In one aspect, compounds according to the invention include type 1 diabetes immunodominant epitopes, such as the IAPP-precursor-derived peptides KLQVFLIVL, KLPAVLLIL, KLNERLAKL, QVFLIVLSV, GILKLQVFL, FLIVLSVAL, or VLSVALNHL (SEQ ID NOs:______). In some aspects, the invention excludes peptides that exhibit MHC class I binding, but do not initiate a type 1 diabetes autoreactive CTL response (e.g., TUM), such compounds are not immunodominant or immunogenic for type 1 diabetes. In some embodiments, the invention also excludes known synthetic peptide autoantigens, or peptides derived from insulin, such as INSL, NRP, NRP-A7, and NRP-V7 that may be capable of initiating a type 1 diabetes autoreactive CTL response in NOD mice. In some embodiments of the invention, isolated peptides that have been disclosed for other uses, for example KLNERLAKL, may be excluded from particular aspects of the invention.

In alternative embodiments, a compound according to the invention can be a non-peptide molecule as well as a peptide or peptide analogue. A peptide or peptide analogue of the invention will generally be nine amino acids in length, although it can range from eight amino acids to ten amino acids in length. In general, a peptide or peptide analogue will be as small as feasible while retaining full biological activity. A non-peptide molecule can be any molecule that exhibits biological activity as described herein. Biological activity can, for example, be measured in terms of ability to elicit an antigen-specific CTL response or to bind specific MHC class I molecules.

An agonist compound will have biological activity if it competes with an IAPP precursor peptide or peptide analogue, as described herein, for binding to a MHC molecule and has similar or enhanced ability to stimulate an antigen-specific, type 1 diabetes autoreactive CTL response when compared to a IAPP precursor peptide or peptide analogue. Generally, an agonist compound will exhibit at least 20% CTL stimulation, or at least 30% to 50% CTL stimulation, or even over 80% or over 100% CTL stimulation when compared to an IAPP precursor peptide or peptide analogue. An agonist compound is useful, for example, in the diagnostic and predictive methods of the invention.

An antagonist compound will have biological activity if it competes with an IAPP precursor peptide or peptide analogue, as described herein, for binding to a MHC molecule and inhibits a type 1 diabetes autoreactive CTL response when compared to the IAPP precursor peptide or peptide analogue. Thus, an antagonist compound will be able to suppress a T-cell mediated or T-cell dependent autoimmune response when administered, or will be able to suppress proliferation of T-cells responsible for or contributing to autoimmune attack on beta cells. Generally, an antagonist compound will exhibit at least 20% CTL inhibition, or at least 30% to 50% CTL inhibition, or even over 80% or over 100% CTL inhibition when compared to an IAPP precursor peptide or peptide analogue.

Compounds can be prepared by, for example, replacing, deleting, or inserting an amino acid residue of an IAPP precursor peptide or peptide analogue, as described herein, with other conservative amino acid residues, i.e., residues having similar physical, biological, or chemical properties, and screening for biological function.

It is well known in the art that some modifications and changes can be made in the structure of a polypeptide without substantially altering the biological function of that peptide, to obtain a biologically equivalent polypeptide. In one aspect of the invention, leader-sequence-derived or IAPP-sequence-derived peptides or epitopes may include peptides that differ from a portion of a native leader, protein or IAPP sequence by conservative amino acid substitutions. The peptides and epitopes of the present invention also extend to biologically equivalent peptides that differ from a portion of the sequence of novel peptides of the present invention by conservative amino acid substitutions. As used herein, the term “conserved amino acid substitutions” refers to the substitution of one amino acid for another at a given location in the peptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

In some embodiments, conserved amino acid substitutions may be made where an amino acid residue is substituted for another having a similar hydrophilicity value (e.g., within a value of plus or minus 2.0), where the following may be an amino acid having a hydropathic index of about −1.6 such as Tyr (−1.3) or Pro (−1.6)s are assigned to amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gln (+0.2); Gly (0); Pro (−0.5); Thr (−0.4); Ala (−0.5); His (−0.5); Cys (−1.0); Met (−1.3); Val (−1.5); Leu (−1.8); Ile (−1.8); Tyr (−2.3); Phe (−2.5); and Trp (−3.4).

In alternative embodiments, conserved amino acid substitutions may be made where an amino acid residue is substituted for another having a similar hydropathic index (e.g., within a value of plus or minus 2.0). In such embodiments, each amino acid residue may be assigned a hydropathic index on the basis of its hydrophobicity and charge characteristics, as follows: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gln (−3.5); Asp (−3.5); Asn (−3.5); Lys (−3.9); and Arg (−4.5).

In alternative embodiments, conserved amino acid substitutions may be made where an amino acid residue is substituted for another in the same class, where the amino acids are divided into non-polar, acidic, basic and neutral classes, as follows: non-polar: Ala, Val, Leu, Ile, Phe, Trp, Pro, Met; acidic: Asp, Glu; basic: Lys, Arg, His; neutral: Gly, Ser, Thr, Cys, Asn, Gln, Tyr.

Conservative amino acid changes can include the substitution of an L-amino acid by the corresponding D-amino acid, by a conservative D-amino acid, or by a naturally-occurring, non-genetically encoded form of amino acid, as well as a conservative substitution of an L-amino acid. Naturally-occurring non-genetically encoded amino acids include beta-alanine, 3-amino-propionic acid, 2,3-diamino propionic acid, alpha-aminoisobutyric acid, 4-amino-butyric acid, N-methylglycine (sarcosine), hydroxyproline, ornithine, citrulline, t-butylalanine, t-butylglycine, N-methylisoleucine, phenylglycine, cyclohexylalanine, norleucine, norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, homoarginine, N-acetyl lysine, 2-amino butyric acid, 2-amino butyric acid, 2,4,-diamino butyric acid, p-aminophenylalanine, N-methylvaline, homocysteine, homoserine, cysteic acid, epsilon-amino hexanoic acid, delta-amino valeric acid, or 2,3-diaminobutyric acid.

In alternative embodiments, conservative amino acid changes include changes based on considerations of hydrophilicity or hydrophobicity, size or volume, or charge. Amino acids can be generally characterized as hydrophobic or hydrophilic, depending primarily on the properties of the amino acid side chain. A hydrophobic amino acid exhibits a hydrophobicity of greater than zero, and a hydrophilic amino acid exhibits a hydrophilicity of less than zero, based on the normalized consensus hydrophobicity scale of Eisenberg et al. (J. Mol. Bio. 179:125-142, 184). Genetically encoded hydrophobic amino acids include Gly, Ala, Phe, Val, Leu, Ile, Pro, Met and Trp, and genetically encoded hydrophilic amino acids include Thr, His, Glu, Gln, Asp, Arg, Ser, and Lys. Non-genetically encoded hydrophobic amino acids include t-butylalanine, while non-genetically encoded hydrophilic amino acids include citrulline and homocysteine.

Hydrophobic or hydrophilic amino acids can be further subdivided based on the characteristics of their side chains. For example, an aromatic amino acid is a hydrophobic amino acid with a side chain containing at least one aromatic or heteroaromatic ring, which may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR, etc., where R is independently (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀)aryl, (C₆-C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe, Tyr, and Tryp, while non-genetically encoded aromatic amino acids include phenylglycine, 2-napthylalanine, beta-2-thienylalanine, 1,2,3,4-tetrahydro-isoquinoline-3-carboxylic acid, 4-chlorophenylalanine, 2-fluorophenylalanine3-fluorophenylalanine, and 4-fluorophenylalanine.

An apolar amino acid is a hydrophobic amino acid with a side chain that is uncharged at physiological pH and which has bonds in which a pair of electrons shared in common by two atoms is generally held equally by each of the two atoms (i.e., the side chain is not polar). Genetically encoded apolar amino acids include Gly, Leu, Val, Ile, Ala, and Met, while non-genetically encoded apolar amino acids include cyclohexylalanine. Apolar amino acids can be further subdivided to include aliphatic amino acids, which is a hydrophobic amino acid having an aliphatic hydrocarbon side chain. Genetically encoded aliphatic amino acids include Ala, Leu, Val, and Ile, while non-genetically encoded aliphatic amino acids include norleucine.

A polar amino acid is a hydrophilic amino acid with a side chain that is uncharged at physiological pH, but which has one bond in which the pair of electrons shared in common by two atoms is held more closely by one of the atoms. Genetically encoded polar amino acids include Ser, Thr, Asn, and Gln, while non-genetically encoded polar amino acids include citrulline, N-acetyl lysine, and methionine sulfoxide.

An acidic amino acid is a hydrophilic amino acid with a side chain pKa value of less than 7. Acidic amino acids typically have negatively charged side chains at physiological pH due to loss of a hydrogen ion. Genetically encoded acidic amino acids include Asp and Glu. A basic amino acid is a hydrophilic amino acid with a side chain pKa value of greater than 7. Basic amino acids typically have positively charged side chains at physiological pH due to association with hydronium ion. Genetically encoded basic amino acids include Arg, Lys, and His, while non-genetically encoded basic amino acids include the non-cyclic amino acids ornithine, 2,3,-diaminopropionic acid, 2,4-diaminobutyric acid, and homoarginine.

It will be appreciated by one skilled in the art that the above classifications are not absolute and that an amino acid may be classified in more than one category. In addition, amino acids can be classified based on known behaviour and or characteristic chemical, physical, or biological properties based on specified assays or as compared with previously identified amino acids. Amino acids can also include bifunctional moieties having amino acid-like side chains.

Conservative changes can also include the substitution of a chemically derivatised moiety for a non-derivatised residue, by for example, reaction of a functional side group of an amino acid. Thus, these substitutions can include compounds whose free amino groups have been derivatised to amine hydrochlorides, p-toluene sulfonyl groups, carbobenzoxy groups, t-butyloxycarbonyl groups, chloroacetyl groups or formyl groups. Similarly, free carboxyl groups can be derivatized to form salts, methyl and ethyl esters or other types of esters or hydrazides, and side chains can be derivatized to form O-acyl or O-alkyl derivatives for free hydroxyl groups or N-im-benzylhistidine for the imidazole nitrogen of histidine. Peptide analogues also include amino acids that have been chemically altered, for example, by methylation, by amidation of the C-terminal amino acid by an alkylamine such as ethylamine, ethanolamine, or ethylene diamine, or acylation or methylation of an amino acid side chain (such as acylation of the epsilon amino group of lysine). Peptide analogues can also include replacement of the amide linkage in the peptide with a substituted amide (for example, groups of the formula —C(O)—NR, where R is (C₁-C₆) alkyl, (C₁-C₆) alkenyl, (C₁-C₆) alkynyl, substituted (C₁-C₆) alkyl, substituted (C₁-C₆) alkenyl, or substituted (C₁-C₆) alkynyl) or isostere of an amide linkage (for example, —CH₂NH—, —CH₂S, —CH₂CH₂—, —CH═CH— (cis and trans), —C(O)CH₂—, —CH(OH)CH₂—, or —CH₂SO—).

The compound can be covalently linked, for example, by polymerisation or conjugation, to form homopolymers or heteropolymers. Spacers and linkers, typically composed of small neutral molecules, such as amino acids that are uncharged under physiological conditions, can be used. Linkages can be achieved in a number of ways. For example, cysteine residues can be added at the peptide termini, and multiple peptides can be covalently bonded by controlled oxidation. Alternatively, heterobifunctional agents, such as disulfide/amide forming agents or thioether/amide forming agents can be used. The compound can also be linked to a lipid-containing molecule or peptide that can enhance a T cell response. The compound can also be constrained, for example, by having cyclic portions.

Peptides or peptide analogues can be synthesised by standard chemical techniques, for example, by automated synthesis using solution or solid phase synthesis methodology. Automated peptide synthesisers are commercially available and use techniques well known in the art. Peptides and peptide analogues can also be prepared using recombinant DNA technology using standard methods such as those described in, for example, Sambrook, et al. (Molecular Cloning: A Laboratory Manual. 2.sup.nd, ed., Cold Spring Harbor Laboratory, Cold Spring Harbor Laboratory Press, Cold Spring Harbor, N.Y., 1989) or Ausubel et al. (Current Protocols in Molecular Biology, John Wiley & Sons, 1994).

Compounds, such as peptides (or analogues thereof) can be identified by routine experimentation by, for example, modifying residues within IAPP precursor peptides; introducing single or multiple amino acid substitutions, deletions, or insertions, and identifying those compounds that retain biological activity, e.g., those compounds that have the ability to modulate a class I-restricted CTL response against pancreatic beta cells. Peptides or analogues that show increased binding affinity to MHC molecules can also be identified. Peptide modifications can also be based on, for example, epitope prediction algorithms, such as those disclosed in MHC Ligands and Peptide Motifs (H. G. Rammensee, J. Bachmann, and S. Stevanovic, Chapman & Hall, 1997; or http://syfpeithi.bmi-heidelberg.com/Scripts/MHCServer.dll/EpPredict.htm) or in Parker et al. (Scheme for ranking potential HLA-A2 binding peptides based on independent binding of individual peptide side-chains, J. Immunol. 152:163, 1994; or http://bimas.dcrt.nih.gov/molbio/hla_bind/).

In general, candidate compounds for prevention or treatment of type 1 diabetes are identified from large libraries of both natural product or synthetic (or semi-synthetic) extracts or chemical libraries according to methods known in the art. Those skilled in the field of drug discovery and development will understand that the precise source of test extracts or compounds is not critical to the method(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available. Alternatively, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A. (Cambridge, Mass.). In addition, natural and synthetically produced libraries of, for example, pancreatic beta cell precursor polypeptides containing leader sequences, are produced, if desired, according to methods known in the art, e.g., by standard extraction and fractionation methods. Furthermore, if desired, any library or compound is readily modified using standard chemical, physical, or biochemical methods.

When a crude extract is found to modulate type 1 diabetes autoreactive CTL, further fractionation of the positive lead extract is necessary to isolate chemical constituents responsible for the observed effect. Thus, the goal of the extraction, fractionation, and purification process is the careful characterization and identification of a chemical entity within the crude extract having cell proliferation, -preventative, or -palliative activities. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using a mammalian type 1 diabetes model.

Candidate test compounds can be first assayed for their ability to inhibit or reduce the proliferative or other response of T lymphocytes that have been shown to be specific to any of the IAPP precursor peptides. The T lymphocytes can be obtained from cell lines or can be isolated from diabetic patients or animal models for type 1 diabetes, using standard techniques. The assays can be performed using standard assays using the isolated T lymphocytes or cell lines as target cells. Candidate test compounds can also be tested for their ability to inhibit or reduce the ability of the T lymphocytes or cell lines to provide help to peptide-specific B lymphocytes in the presence of any of the IAPP precursor peptides. Candidate test compounds can also be chosen on the basis of their ability to bind to MHC molecules on relevant antigen presenting cells and to compete with binding of the IAPP precursor peptide epitopes. Test compounds that modulate T cell responses or bind to MHC molecules can then be used for further analysis.

Test compounds identified as being modulators of CTL responses can be further tested in animal models of diabetes, such as the NOD mouse, using standard techniques, for their ability to reduce diabetes in a suitable animal model. Another in vivo assay can be to administer a peptide, as an IAPP precursor peptide, that can induce earlier onset of diabetes in NOD mice, and assay candidate test compounds for their ability to inhibit or reverse the disease. Test compounds can also be assayed for their ability to bind to human MHC molecules, but inhibit T lymphocyte proliferation. Appropriate cells can be obtained from the peripheral blood of diabetic patients and healthy controls.

Antibodies

The compounds of the invention can be used to prepare antibodies to IAPP precursor peptides or analogues thereof, optionally in combination with an MHC molecule, using standard techniques of preparation as, for example, described in Harlow and Lane (Antibodies; A Laboratory Manual, Cold Spring Harbor Laboratory, Cold Spring Harbor, N.Y., 1988), or known to those skilled in the art. Antibodies can be tailored to minimise adverse host immune response by, for example, using chimeric antibodies contain an antigen binding domain from one species and the Fc portion from another species, or by using antibodies made from hybridomas of the appropriate species. MHC/antigen-specific antibodies can be used, for example, to directly modulate type 1 diabetes autoreactive CTL responses.

Pharmaceutical Compositions, Dosages, and Administration

Compounds of the invention can be provided alone or in combination with other compounds (for example, small molecules, peptides, or peptide analogues), in the presence of a liposome, an adjuvant, or any pharmaceutically acceptable carrier, in a form suitable for administration to humans.

Conventional pharmaceutical practice may be employed to provide suitable formulations or compositions to administer the compounds to patients suffering from or presymptomatic for type 1 diabetes. Any appropriate route of administration may be employed, for example, parenteral, intravenous, subcutaneous, intramuscular, intracranial, intraorbital, ophthalmic, intraventricular, intracapsular, intraspinal, intracisternal, intraperitoneal, intranasal, aerosol, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found in, for example, “Remington's Pharmaceutical Sciences” (18^{th} edition), ed. A. Gennaro, 1990, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

If desired, treatment with a compound according to the invention may be combined with more traditional therapies for the disease such as, for example, surgery, including pancreas or pancreatic islet transplantation, or insulin administration.

For therapeutic or prophylactic compositions, the compounds are administered to an individual in an amount sufficient to stop or slow the destruction of beta cells, or to stimulate the growth of new beta cells. Amounts considered sufficient will vary according to the specific compound used, the mode of administration, the stage and severity of the disease, the age, sex, and health of the individual being treated, and concurrent treatments. As a general rule, however, dosages can range from about 1 μg to about 100 mg per kg body weight of a patient for an initial dosage, with subsequent adjustments depending on the patient's response, which can be measured, for example by determining specific CTL activity in the patient's peripheral blood.

In the case of vaccine formulations, an immunogenically effect amount of a compound of the invention can be provided, alone or in combination with other compounds, with an adjuvant, for example, Freund's incomplete adjuvant or aluminum hydroxide. The compound may also be linked with a carrier molecule, such as bovine serum albumin or keyhole limpet hemocyanin to enhance immunogenicity.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

The following examples are intended to illustrate various embodiments and aspects of the invention, and do not limit the invention in any way.

### General Methods Used

ELISPOT Assays

ELISPOT assays can be performed to determine the effectiveness of various compounds as epitopes for diabetes. 96 well plates are coated with anti-mouse or anti-human interferon-gamma antibody (5 μg/ml in phosphate buffered saline (PBS)) and incubated at 4° C. overnight. The plates are washed with PBS/Tween 6 times, after which cell culture medium containing fetal calf serum is added at room temperature for 1-2 hours to minimize non-specific binding to the ELISPOT plate.

The effector cells (CTL) of the ELISPOT assay are contained within spleen cells from NOD mice aged 10 weeks. The spleens from these mice contain a proportion of autoreactive CTL destined to destroy pancreatic beta cells. Non-obese diabetic (NOD) mouse spleen effector cells are co-incubated at a concentration of 5×10⁵ cells/well in medium with antigen presenting cells (“APCs,” for example, irradiated NOD spleen cells, RMAS-Kd or P815 cells) that are pre-coated overnight with peptides of interest (for example, TV1, TUM, INSL, or NRP-A7).

Effector cells that recognise antigen presenting cells in association with peptide will be triggered to secrete interferon-gamma, which can then be captured by the interferon-gamma antibody on the ELISPOT plate. The cells are removed and secondary antibody is added, then a colour detection method is employed to visualise the cells that have secreted interferon-gamma. Accordingly, the effector cells are transferred to an ELISPOT plate and cultured for 24-48 hours. The ELISPOT plate is then washed 6 times with PBS/Tween, biotinylated-anti-mouse interferon gamma antibody is added, and the plate is incubated for 3 hours at room temperature. The plate is washed 10 times with PBS/Tween, after which streptavidin alkaline phosphatase is added. The plate is incubated for 1 hour at room temperature. The plate is washed 10 times with PBS/Tween again, a substrate for alkaline phosphatase is added, and the plate is incubated at room temperature until spots are observed. The plate is washed with water and dried, then the spots are counted to determine the proportion of T cells that have recognised the target cells/epitope and have been activated to secrete interferon-gamma. Similarly, human ELISPOT assays are performed using antibodies that recognize human IFN-gamma and effector cells obtained from the peripheral blood of diabetic patients.

MHC Class I Stabilization Assay

RMAS-Kd cells (murine antigen presenting cells) are incubated overnight at room temperature (26° C.). A peptide (for example, TUM, V7, INSL, TV1, TV2, or TV3) is added and the cells are incubated at room temperature for 1 hour, and then incubated at 37° C. for 3 hours. The cells are washed 3 times with 0.3% bovine serum albumin-phosphate buffer solution (BSA-PBS), and then stained with fluorescently labelled anti-mouse H-2Kd-FITC antibody. Fluorescent-activated cell sorting (FACS) analysis is performed to count the percentage of cells showing positive staining.

MHC Class I Tetramers

MHC class I tetramers (for example, H-2Kd tetrameric complexes) are synthesised as previously described by Altman et al. (Science 274:94-96, 1996). MHC heavy chain, modified by the addition of a C-terminal biotinylation site, and beta 2 microglobulin are expressed separately in E. coli (BL21, Stratagene, La Jolla, Calif., U.S.A.). Peptides (KYNKANWFL (NRP, SEQ ID NO:______), KYNKANAFL (NRP-A7, SEQ ID NO:______), KYQAVTTTL (TUM, SEQ ID NO:______) are synthesised on a Perkin-Elmer-ABI 431A by the NAPS Unit, University of British Columbia, BC, Canada. Purified heavy chain, beta 2 microglobulin and peptide are refolded at 4° C. for 48 hours in a TRIS-based buffer, and the refolded product is concentrated, biotinylated using BirA enzyme (Avidity, Denver, Colo., U.S.A.) in the presence of biotin, ATP and Mg++ and purified by FPLC. Streptavidin-phycoerythrin conjugate is added in a 1:4 molar ratio and the tetrameric product is concentrated to approximately 1 mg/ml. The tetramer concentration is calculated based upon the concentration of biotinylated protein, quantified by Bradford assay, prior to addition of streptavidin.

Single cell suspensions of lymphocytes are incubated with 1 μl of a 1 mg/ml solution of MHC tetramer for 2-3 hours at 4° C. in FACS staining buffer (HBSS/FBS 2%/Sodium Azide 0.01%/EDTA 100 mM). The cells are centrifuged and washed three times before staining with antibody to CD8.

In Vivo Protocol

Peptides are administered to female NOD mice in accordance with standard laboratory methods.

### EXAMPLE 1

Autoreactive T Cells Recognise the TV1 Epitope

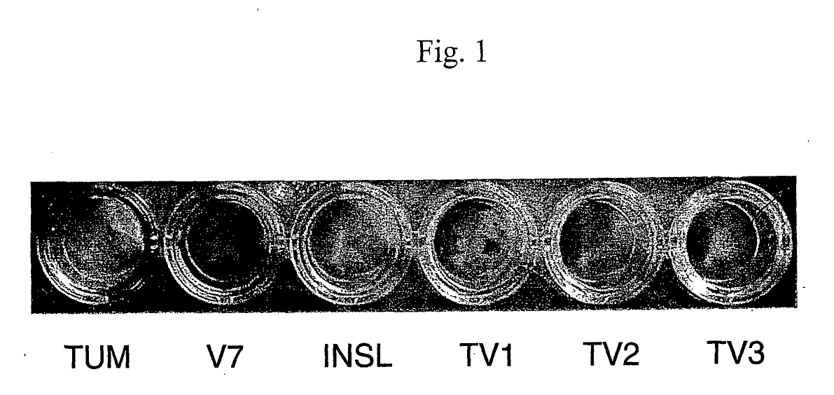

ELISPOT assays were performed to illustrate the effectiveness of various peptides as epitopes for diagnosis of diabetes. NOD spleen cells were assayed to determine the frequency of CTL that react to the murine IAPP precursor peptide mTV1 (KLPAVLLIL, SEQ ID NO:______). As shown in FIG. 1, an interferon-gamma ELISPOT assay was used to show that murine autoreactive T cells recognise mTV1. Autoreactive T cells recognise in particular the peptide epitopes NRP-V7 (KYNKANVFL, SEQ ID NO:______) and mTV1. The peptide NRP-V7 was used as a positive control and is a previously described synthetic autoepitope (automimotope) of NOD mice (Amrani et al., 2000). NRP-V7 was not derived from any endogenous mammalian protein sequence but was identified by screening combinatorial peptide libraries. The peptide TUM was used as a negative control. TUM is derived from an endogenous tumour peptide not known to participate in type 1 diabetes but known to bind to the MHC class I molecule, H-2Kd. MTV1 is an epitope of the invention derived from the leader sequence of preproIAPP. TV2 (KYPAVLLIL, SEQ ID NO:______) is a modified version of TV1, containing a leucine (L) to tyrosine (Y) substitution at position 2 of the peptide. INSL (LYLVCGERG, SEQ ID NO:______) is an insulin-derived peptide believed to be an autoepitope in NOD mice (Wong et al., 2001 and 2002). TV3 (RLLPLLALL, SEQ ID NO:______) is a peptide derived from the mouse insulin protein. The results show that spleen and islet cells derived from pre-diabetic NOD mice contain a high frequency of CTL that recognise TV-1 and secrete interferon gamma in response to TV-1 binding. Interferon-gamma is also secreted by autoreactive spleen cells from NOD mice in response to a known autoepitope, NRP-V7, but not in response to a negative control peptide, TUM. Thus, in one aspect of the invention, TV1 provides an immunodominant epitope for diagnostic detection of CTL in type 1 diabetes.

### EXAMPLE 2

Murine Antigen Presenting Cells Express MHC Class I Molecules on their Cell Surface in Association with TV1 Peptide

FACS analysis was performed to quantify the percentage/proportion of antigen presenting cells (RMAS-Kd) that express stable MHC class I on their cell surface, in association with the TV1 peptide. RMAS-Kd is a transporter for antigen presentation (TAP)-deficient RMAS cell line transfected with the mouse MHC class I molecule, H-2Kd. Because the cell line is TAP deficient it cannot assemble MHC class I (in this case H-2Kd) complexes stably but requires surface stabilisation by exogenous peptides that can bind with the H-2Kd heavy chain and beta-2 microglobulin. The cell line was stained for MHC class I expression with or without TV1 peptide.

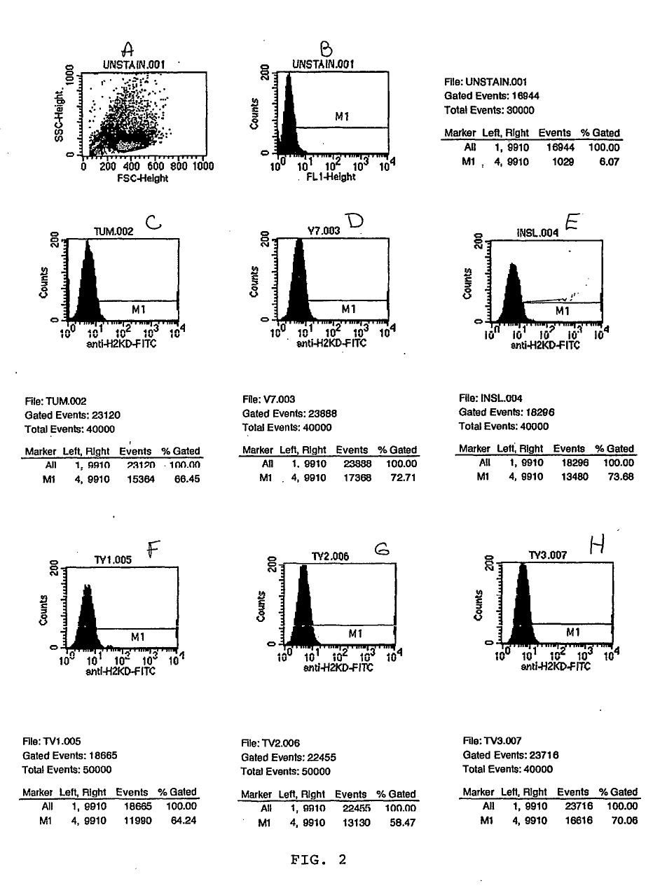

As shown in FIGS. 2A-H, a significant percentage of antigen presenting cells (RMAS-Kd) are able to express stably bound TV1 peptide in association with H-2Kd MHC class I on the cell surface. Where RMAS-Kd cells were incubated in the absence of peptide (FIG. 2B_?), a result of 6.07% binding was obtained. This constitutes the background amount of MHC class I expressed on the surface of antigen presenting cells in the absence of a bound, stabilising peptide. For the peptide samples TUM (FIG. 2C), V7 (FIG. 2D), INSL (FIG. 2E), all known to be presented by the H-2Kd Class I molecule, the results obtained were 66.45%, 72.71%, and 73.68%. For the TV1 (FIG. 2F), TV2 (FIG. 2G), and TV3 (FIG. 2H) peptides, the results obtained were 64.24%, 58.47%, and 70.06%, respectively.

The results show that a significant percentage of antigen presenting cells that express the murine MHC class I molecule, H-2Kd, bind and present TV1 on the cell surface, indicating that the peptide binds stably to H-2Kd, since in the absence of peptide binding, MHC molecules are inherently unstable.

### EXAMPLE 3

Autoreactive Tetramer Positive T Cells Accumulate within the Pancreatic Islets of NOD Mice

Islets were isolated from the pancreatic tissue of NOD mice by injection of collagenase into the common bile duct. The pancreas was removed and incubated at 37° C. for ˜20 min to allow digestion of the exocrine tissue away from the islets. The islet fraction was separated by dextran gradient centrifugation and then the islets were hand-picked. Mice were grouped by age and the proportion of autoreactive T cells in the islets recognising the previously described autoepitopes, NRP-V7 and INSL or control peptide, TUM, in a complex with MHC class I tetramers, was determined.

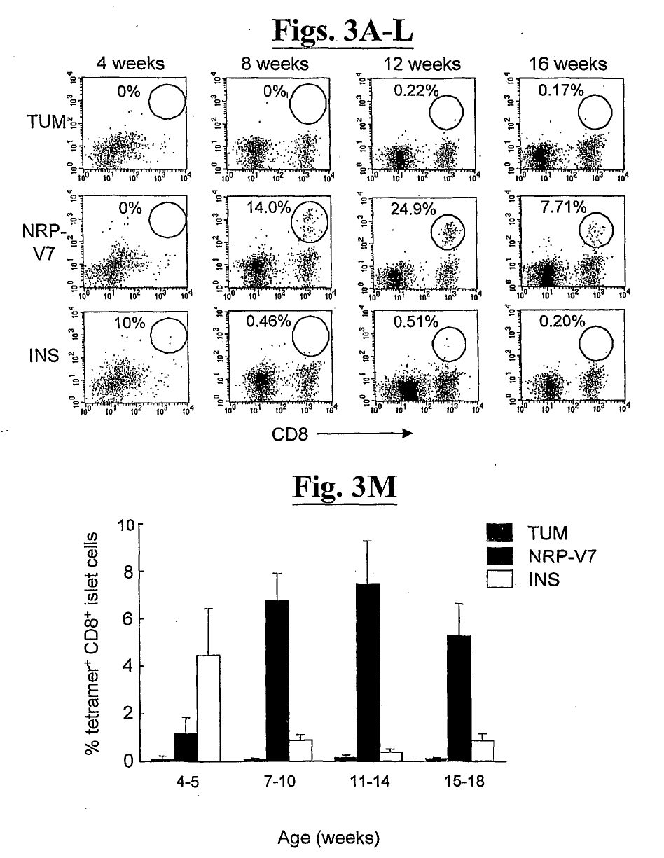

FIGS. 3A-L show flow cytometry data demonstrating that autoreactive (tetramer-positive) T cells accumulate within the pancreatic islets of NOD mice as they age and develop clinical disease. FIG. 3M is a summary of the raw flow cytometry data shown in FIGS. 3A-L.

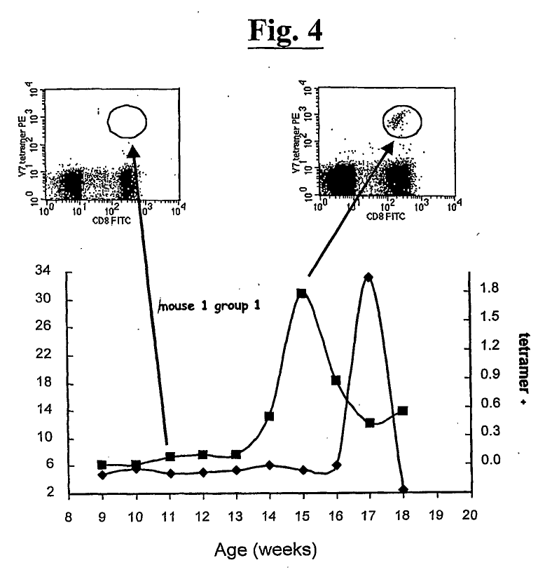

FIG. 4 is an example of the detection of autoreactive (tetramer-positive) T cells in peripheral blood in a single mouse. A single female NOD mouse was followed weekly for blood glucose (diamonds, black line) and blood tetramer-positive frequency (squares, grey line) from 9 to 18 weeks of age. The arrows correspond with the actual flow cytometry data illustrating tetramer negative (A) and tetramer positive (B) populations of T cells. In this case, a peak in the proportion/number of autoreactive T cells is noted at 15 weeks.

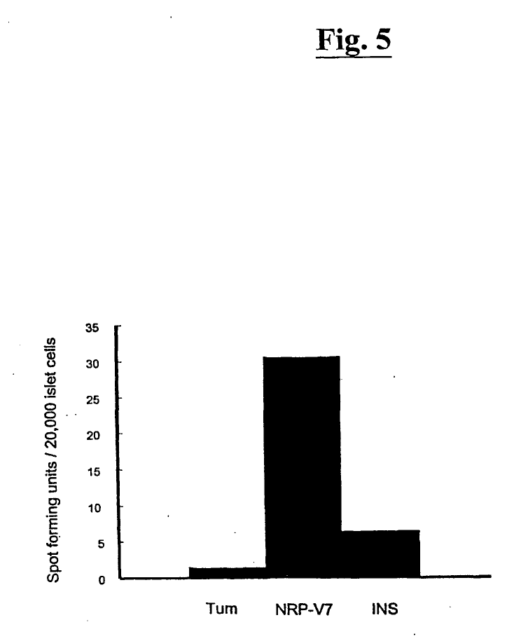

FIG. 5 shows that the autoreactive T cells in NOD pancreatic islets secrete interferon-gamma, in an elispot assay, in response to the previously identified autoepitope NRP-V7, but minimally in response to the autoepitope INSL and not to the peptide TUM.

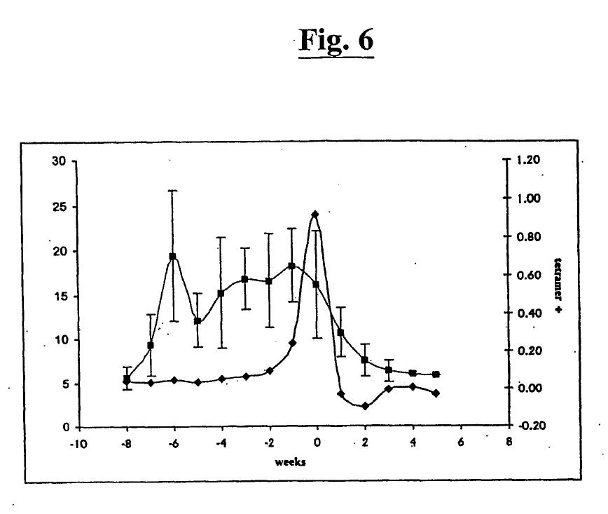

FIG. 6 shows pooled data from all hyperglycaemic mice, normalised to the time at which hyperglycaemia appears (Time 0). Glucose concentration prior to, during, and after onset of hyperglycaemia is shown (diamonds, black line). The percentage of CD8 expressing cells that are NRP-V7 tetramer positive is also shown (squares, grey line). These data show that autoreactive T cells appear, on average, 6 weeks prior to the onset of hyperglycaemia (clinical disease) in NOD mice. Normal glucose concentration is about 6 mM, and elevates to greater than 17 mM during onset of clinical diabetes.

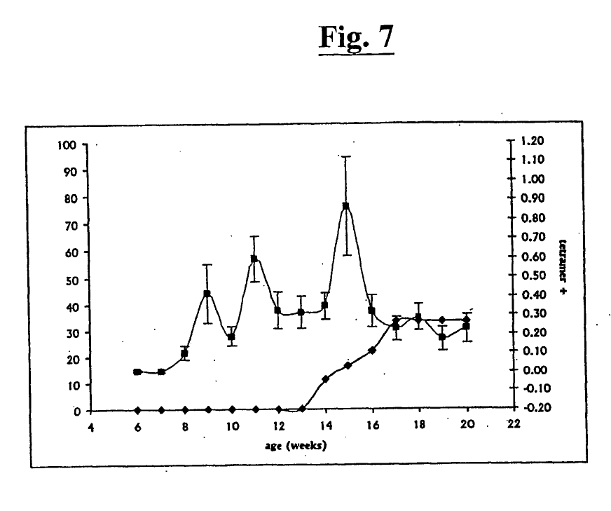

FIG. 7 shows that autoreactive T cells may appear in waves or in cyclical fashion prior to onset of hyperglycaemia. The percentage of mice that are diabetic (diamonds, black line) and the percentage of CD8 expressing cells that are NRP-V7 tetramer positive (squares, grey line) are shown. NRP-V7-specific T cells appear in cycles of increasing magnitude at regular intervals in female NOD mice prior to onset of clinical diabetes (n=18).

FIGS. 3A-M to 7 demonstrate that the appearance of autoimmune T cells precedes the development of diabetes (FIGS. 1, 2A-H, 4, 5) and that the autoreactive CTL can be detected and quantified in islet cells (FIG. 1) and in peripheral blood (FIGS. 2A-H, 4, 5). The discovery that autoreactive CTL can be readily visualised and quantified in peripheral blood, using, for example, MHC class 1 tetramers complexed to a relevant peptide epitope, has significant implications for human disease, since blood is an easily accessible organ. In addition, FIGS. 3A-M demonstrate that the detected cells are functional since they secrete interferon-gamma in a peptide epitope-specific manner.

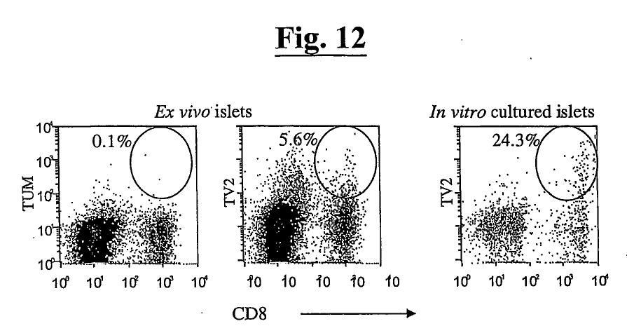

FIG. 12 shows flow cytometry dot plots showing CTL from pancreatic islets of NOD mice recognize MTV2. Islet cells were stained directly ex vivo or following in vitro culture with H-2Kd tetramers bearing MTV2 or TUM (negative control) to determine the proportion of CD8+ tetramer+T cells present. This illustrates that epitopes of the invention may be used in various ways to diagnose type 1 diabetes, for example by detecting or isolating autoreactive T cells.

### EXAMPLE 4

Peptide Immunisation of NOD Mice

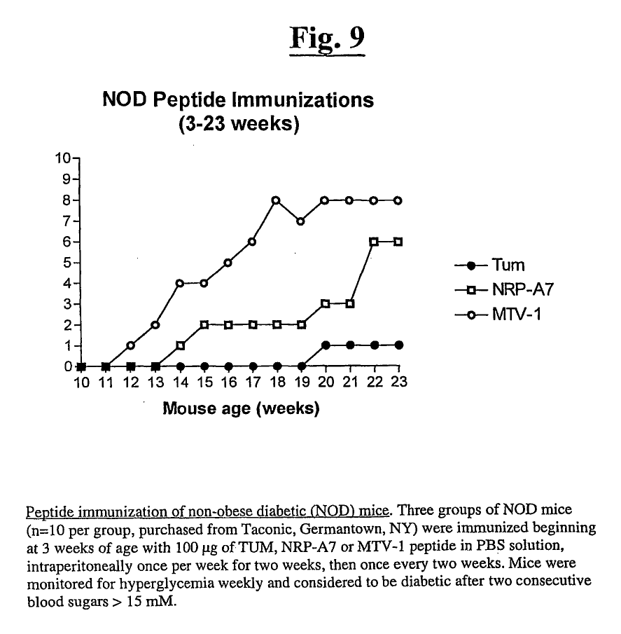

Three groups of NOD mice (n=10 per group, purchased from Taconic, Germantown, N.Y., U.S.A.) were immunised beginning at three weeks of age with 100 μg of TUM, NRP-A7, or TV1 (MTV-1) peptide in PBS solution, intraperitoneally once per week for two weeks, then once every two weeks. Mice were monitored for hyperglycaemia weekly and considered to be diabetic after two consecutive blood sugars >15 mM. FIG. 9 shows progressive diabetic onset in mice immunized with TV-1, so that by week 18, 80% of the immunized mice were considered diabetic, compared to 20% of mice immunized with the positive control peptide NRP-A7 and none of the mice immunized with the negative control peptide TUM. The data indicate that administration of TV1 causes diabetes to occur earlier in NOD mice, providing an animal model of disease.

### EXAMPLE 5

Autoreactive T Cells from Diabetic Patients Recognise HTV1 Peptide

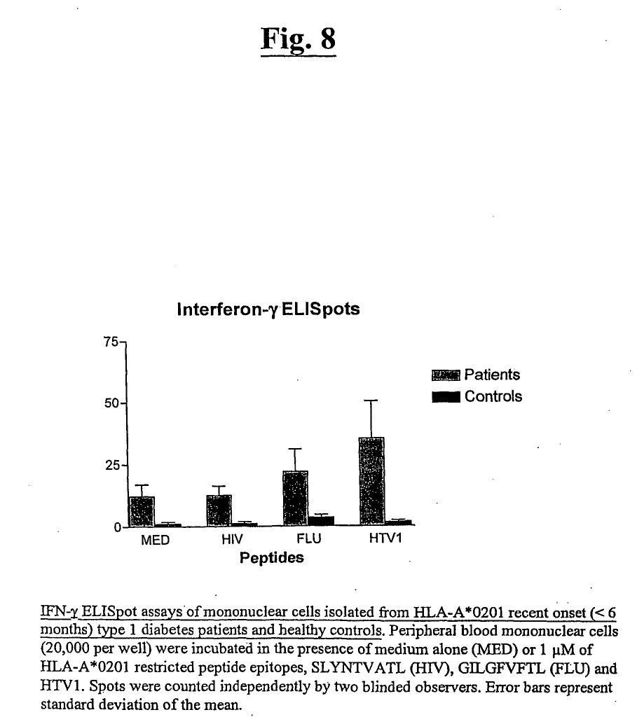

Interferon gamma ELISPOT assays of mononuclear cells isolated from HLA-A*0201 recent onset (<6 months) type 1 diabetes patients and healthy controls were done (FIG. 8). Peripheral blood mononuclear cells (PBMC; 20,000 per well) were incubated in the presence of medium alone (MED) or 1 μM of HLA-A*0201 restricted peptide epitopes, SLYNTVATL (HIV; SEQ ID NO:______), GILGFVFTL (FLU; SEQ ID NO:______), and HTV1. Spots were counted independently by two blinded observers. FIG. 8 is a bar graph of these ELISPOT results, showing that elevated HTV1 autoreactivity may be used as a diagnostic indicator of disease in type 1 diabetes, including recent onset patients.

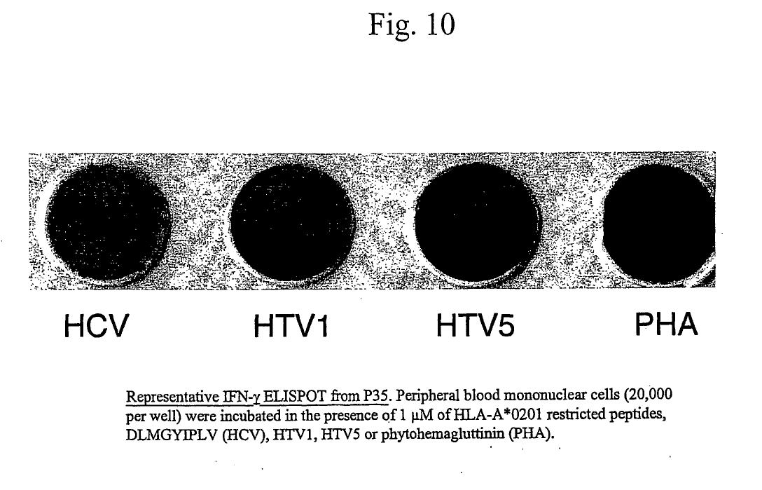

FIG. 10 shows a representative interferon gamma ELISPOT done using PBMC (20,000 per well) from a diabetic patient with residual beta cell function (C-peptide 480 pmol/L, normal 165-1000 pmol/L) and using 1 μM of HLA-A*0201 restricted peptide epitopes DLMGYILV (HCV; SEQ ID NO:______), HTV1, HTV5, or phytohemagluttinin (PHA). The results show that the epitopes of the invention, such as HTV1 and HTV5, may be used as diagnostic indicators of disease in type 1 diabetes patients, including long-term patients, such as patients having residual beta cell function.

### EXAMPLE 6

Human Antigen Presenting Cells Express MHC Class I Molecules on their Cell Surface in Association with HTV1

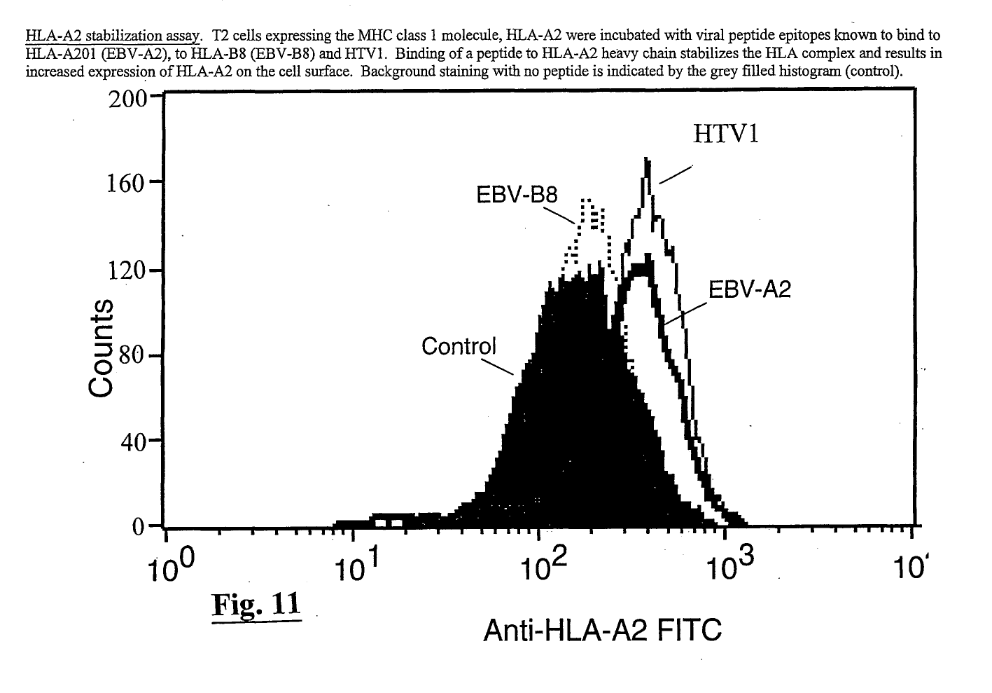

T2 cells expressing the MHC class I molecule HLA-A2 were incubated with HTV1 and with viral peptide epitopes known to bind to HLA-A201 (EBV-A2: GLCTLVAML, SEQ ID No.______), HLA-B8 (EBV-B8: RAKFKQLL, SEQ ID No.______). FACS analysis was performed to assay the extent of epitope binding, by segregating cells binding to the peptides. Binding of a peptide to a HLA-A2 heavy chain stabilises the HLA complex and results in increased expression of HLA-A2 on the cell surface (FIG. 11). Background staining with no peptide is indicated by the grey filled histogram (control). These results illustrate that epitopes of the invention, such as HTV1, bind to HLA-A2, and that epitopes of the invention may be used to isolate or identify cells that bind to epitopes of the invention, such as autoreactive T cells.

### OTHER EMBODIMENTS

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to”, and the word “comprises” has a corresponding meaning. Citation of references herein shall not be construed as an admission that such references are prior art to the present invention. All publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

### REFERENCES

The following documents are incorporated herein by reference:
Amrani A, Verdaguer J, Serra P, Tafuro S, Tan R, Santamaria P., 2000, Progression of autoimmune diabetes driven by avidity maturation of a T-cell population. Nature 406:739-42.
Daniel, D. and Wegmann, D. R., 1996, Protection of nonobese diabetic mice from diabetes by intranasal or subcutaneous administration of insulinpeptide B-(9-23). PNAS 93(2): 956-960.
Ekawa, K., Nishi, M., Ohagi, S., Sanke, T. and Nanjo, K., 1997, “Cloning of mouse islet amyloid polypeptide gene and characterization of its promoter” J. Mol. Endocrinol. 19 (1), 79-86).
Hoppener, J. W., Oosterwijk, C., Visser-Vemooy, H. J., Lips, C. J. and Jansz, H. S., 1992, “Characterization of the human islet amyloid polypeptide/amylin gene transcripts: identification of a new polyadenylation site”, Biochem. Biophys. Res. Commun. 189 (3), 1569-1577.
Kapturczak M H, Flotte T, Atkinson M A., 2001, “Adeno-associated virus (AAV) as a vehicle for therapeutic gene delivery: improvements in vector design and viral production enhance potential to prolong graft survival in pancreatic islet cell transplantation for the reversal of type 1 diabetes” Curr Mol Med; 1(2):245-58.
Wong F S, Moustakas A K, Wen L, Papadopoulos G K, Janeway C A Jr., 2002, “Analysis of structure and function relationships of an autoantigenic peptide of insulin bound to H-2K(d) that stimulates CD8 T cells in insulin-dependent diabetes mellitus.”, Proc Natl Acad Sci USA 16;99(8):5551-6.
Wong F S, Moustakas A K, Wen L, Papadopoulos G K, Janeway C A Jr., 2001, “Analysis of Structure and Function of the binding of an autoantigenic peptide of insulin to CD8 T cells in diabetes.” Abstract: 572, 37th Annual Meeting of the European Association for the Study of Diabetes, 9-13 Sep. 2001, Glasgow, United Kingdom.
Yamaoka T., 2001, “Gene therapy for diabetes mellitus” Curr Mol Med; 1(3):325-37.

1. A diagnostic method for providing information about a type 1 diabetes disease state in a human patient, the method comprising contracting a sample comprising a T lymphocyte from the patient with a diagnostic compound comprising a diagnostic epitope of Formula I:
Z₁-X₋₁-X₁-X₂-X₃-X₄-X₅-X₆-X₇-X₈-X₉-X₊₁-Z₂; (I)
wherein
X₋₁ at each occurrence is independently selected from any amino acid or is absent;
X₁ is a hydrophilic amino acid selected from the group consisting of T, H, E, Q, N, R, S or K;
X₂ is Leu, Met, Ile, Phe, Ala, Gly, Val, or Trp;
X₃ is any amino acid;
X₄ is any amino acid;
X₅ is any amino acid;
X₆ is any amino acid;
X₇ is any amino acid;
X₈ is any amino acid;
X₉ is Leu, Ile, or Val;
X₊₁ is any amino acid or is absent;
Z₁ is H₂N—, RHN— or, RRN—;
Z₂ is —C(O)OH, —C(O)R, —C(O)OR, —C(O)NHR, —C(O)NRR;
R at each occurrence is independently selected from (C₁-C₆) alkyl, (C₁-C₆) alkenyl, (C₁-C₆) alkynyl, substituted (C₁-C₆) alkyl, substituted (C₁-C₆) alkenyl, or substituted (C₁-C₆) alkynyl;
wherein “−” is a covalent linkage;
wherein X₋₁ and X₊₁ cannot both be present; and,
wherein the diagnostic compound binds to the T lymphocyte with an affinity that is at least as great as the affinity when the diagnostic epitope is KLQVFLIVL (HTV-1, SEQ ID NO:1) or KLNERLAKL (HTV-5, SEQ ID NO:2).

2. A method of modulating an immune response in a human patient in need of such treatment, the method comprising contacting a T lymphocyte from the patient with an effective amount of a therapeutic compound comprising a therapeutic epitope of Formula I:
Z₁-X₋₁-X₁-X₂-X₃-X₄-X₅-X₆-X₇-X₈-X₉-X₊₁-Z₂; (I)
wherein
X₋₁ at each occurrence is independently selected from any amino acid or is absent;
X₁ is a hydrophilic amino acid selected from the group consisting of T, H, E, Q, N, R, S or K;
X₂ is Leu, Met, Ile, Phe, Ala, Gly, Val, or Trp;
X₃ is any amino acid;
X₄ is any amino acid;
X₅ is any amino acid;
X₆ is any amino acid;
X₇ is any amino acid;
X₈ is any amino acid;
X₉ is Leu, Ile, or Val;
X₊₁, is any amino acid or is absent;
Z₁ is H₂N—, RHN— or, RRN—;
Z₂ is —C(O)OH, —C(O)R, —C(O)OR, —C(O)NHR, —C(O)NRR;
R at each occurrence is independently selected from (C₁-C₆) alkyl, (C₁-C₆) alkenyl, (C₁-C₆) alkynyl, substituted (C₁-C₆) alkyl, substituted (C₁-C₆) alkenyl, or substituted (C₁-C₆) alkynyl;
wherein “−” is a covalent linkage;
wherein X₋₁ and X₊₁ cannot both be present; and,
wherein the therapeutic compound binds to the T lymphocyte with an affinity that is at least as great as the affinity when the therapeutic epitope is KLQVFLIVL (HTV-1, SEQ ID NO:1) or KLNERLAKL (HTV-5, SEQ ID NO:2).

3-30. (canceled)

31. The method of claim 1, wherein the method is carried out in vivo or in vitro.

32. (canceled)

33. The method of claim 31, wherein the method is carried out at a first time-point and repeated at a second time-point.

34. The method of claim 31, wherein the T lymphocyte is a cytotoxic T lymphocyte.

35. The method of claim 31, wherein the compound further comprises a major histocompatibility complex class I molecule.

36-38. (canceled)

39. The method of claim 31, wherein the epitope is substantially identical to a sequence selected from the group consisting of KLQVFLIVL SEQ ID NO:1), KLPAVLLIL (SEQ ID NO:3), KLNERLAKL SEQ ID NO:2), QVFLIVLSV (SEQ ID NO:6), GILKLQVFL (SEQ ID NO:7), FLIVLSVAL (SEQ ID NO:8) and VLSVALNHL (SEQ ID NO:9).

40-43. (canceled)

44. The method of claim 31, wherein the epitope is substantially identical to a sequence selected from the group consisting of KLQVFLIVL (SEQ ID NO:1), KLPAVLLIL (SEQ ID NO:3), KLNERLAKL (SEQ ID NO:2), QVFLIVLSV (SEQ ID NO:6), GILKLQVFL (SEQ ID NO:7), FLIVLSVAL (SEQ ID NO:8) and VLSVALNHL (SEQ ID NO:9).

45. The method of claim 1, wherein the epitope consists essentially of eight to ten amino acids of an IAPP leader peptide.

46. (canceled)

47. The method of claim 2 wherein the therapeutic compound is provided in combination with an antigen presenting cell.

48-49. (canceled)

50. The method of claim 47, wherein the antigen presenting cell express a nucleotide sequence encoding the compound.

51. The method of claim 1, wherein the sample is a peripheral blood sample.

52. The method of claim 2 further comprising the step of determining the proportion of type 1 diabetes autoreactive T lymphocytes present in the sample.

53. A substantially pure compound that binds to an autoreactive T lymphocyte from a subject having type 1 diabetes, the compound having an epitope of Formula I:
Z₁-X₋₁-X₁-X₂-X₃-X₄-X₅-X₆-X₇-X₈-X₉-X₊₁-Z₂; (I)
wherein
X₋₁ at each occurrence is independently selected from any amino acid or is absent;
X₁ is a hydrophilic amino acid selected from the group consisting of T, H, E, Q, N, R, S or K;
X₂ is Leu, Met, Ile, Phe, Ala, Gly, Val, or Trp;
X₃ is any amino acid;
X₄ is any amino acid;
X₅ is any amino acid;
X₆ is any amino acid;
X₇ is any amino acid;
X₈ is any amino acid;
X₉ is Leu, Ile, or Val;
X₊₁ is any amino acid or is absent;
Z₁ is H₂N—, RHN— or, RRN—;
Z₂ is —C(O)OH, —C(O)R, —C(O)OR, —C(O)NHR, —C(O)NRR;
R at each occurrence is independently selected from (C₁-C₆) alkyl, (C₁-C₆) alkenyl, (C₁-C₆) alkynyl, substituted (C₁-C₆) alkyl, substituted (C₁-C₆) alkenyl, or substituted (C₁-C₆) alkynyl;
wherein “−” is a covalent linkage;
wherein X₋₁ and X₊₁ cannot both be present; and,
wherein the compound binds to the T lymphocyte with an affinity that is at least as great as the affinity when the epitope is KLQVFLIVL (HTV-1, SEQ ID NO:1) or KLNERLAKL (HTV-5, SEQ ID NO:2) or KLPAVLLIL (MTV1, SEQ ID NO:3).

54-81. (canceled)

82. The compound of claim 53, wherein the epitope is substantially identical to a sequence selected from the group consisting of KLQVFLIVL (SEQ ID NO:1), KLPAVLLIL (SEQ ID NO:3), KLNERLAKL (SEQ ID NO:2), QVFLIVLSV (SEQ ID NO:6), GILKLQVFL (SEQ ID NO:7), FLIVLSVAL (SEQ ID NO:8) and VLSVALNHL (SEQ ID NO:9).

83-88. (canceled)

89. A method for isolating a T lymphocyte, the method comprising isolating T lymphocytes that bind to the compound of claim 82.

90. A method of identifying compounds that are immunogenic in type 1 diabetes, the method comprising isolating compounds that bind to a T cell receptor from the T lymphocyte of claim 89.

91. (canceled)

92. Isolated T lymphocytes that bind specifically to an epitope that is substantially identical to a sequence selected from the group consisting of KLQVFLIVL (SEQ ID NO:1), KLPAVLLIL (SEQ ID NO:3), KLNERLAKL (SEQ ID NO:2), QVFLIVLSV (SEQ ID NO:6), GILKLQVFL (SEQ ID NO:7), FLIVLSVAL (SEQ ID NO:8) and VLSVALNHL (SEQ ID NO:9).

93-95. (canceled)

96. A substantially pure compound that is immunodominant for type 1 diabetes, wherein said compound is derived from a pancreatic beta cell leader polypeptide and comprises a major histocompatibility complex class I binding motif.

97-98. (canceled)

99. The compound of claim 96, wherein said compound consists essentially of an amino acid sequence selected from the group consisting of KLQVFLIVL (SEQ ID NO:1), KLPAVLLIL (SEQ ID NO:3), KLNERLAKL (SEQ ID NO:2), QVFLIVLSV (SEQ ID NO:6), GILKLQVFL (SEQ ID NO:7), FLIVLSVAL (SEQ ID NO:8) and VLSVALNHL (SEQ ID NO:9).

100-103. (canceled)

104. A pharmaceutical composition comprising a compound according to claim 99 in combination with a physiologically acceptable carrier.

105. An isolated antibody or T cell receptor that specifically binds to a compound according to claim 99.

106. (canceled)

107. The method claim 2 wherein the method is carried out in vivo or in vitro.

108. The method of claim 2, wherein the epitope consists essentially of eight to ten amino acids of an IAPP leader peptide.
